(19) United States
(12) Patent Application Publication
(10) Pub. No.: US 2004/0197312 A1
(43) Pub. Date: Oct. 7, 2004

(54) Cytokine-expressing cellular vaccine combinations

(76) Inventors: Marina Moskalenko, Pacifica, CA (US); Betty Li, San Francisco, CA (US); Sandra Aung, San Francisco, CA (US); Rodney Prell, Richmond, CA (US); Jennifer Creson, San Mateo, CA (US); Karin Jooss, San Francisco, CA (US)

Correspondence Address: Supervisor, Patent Prosecution Services, PIPER RUDNICK LLP, 1200 Nineteenth Street, N.W., Washington, DC, 20036-2412, US

(21) Appl. No.: 10/404,662

(22) Filed: Apr. 2, 2003

(51) Int. Cl. A61K 48/00; A61K 39/395; A61K 38/19

(52) U.S. Cl. 424/93.21; 424/145.1; 424/85.1

(57) ABSTRACT

The present invention in all of its associated aspects provides improved methods and compositions for treating cancer in a mammal based on the administration of the combination of a cytokine-expressing cellular vaccine and at least one additional cancer therapeutic agent or treatment to a patient with cancer, wherein administration of the combination results in enhanced therapeutic efficacy relative to administration of components thereof as a monotherapy.

### FIELD OF THE INVENTION

The present invention relates to a method of altering an individual's immune response to a target cancer antigen or antigens. More particularly, the invention is concerned with administering a cytokine-expressing cellular vaccine combination comprising a cytokine-expressing cellular vaccine (e.g., GVAX) and at least one additional cancer therapeutic agent or treatment to a patient with cancer. The invention also relates to compositions comprising a cytokine-expressing cellular vaccine and at least one additional cancer therapeutic agent or treatment.

### BACKGROUND OF THE INVENTION

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for treatment of cancer including chemotherapy, surgery, radiation therapy and cellular therapy, have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity.

The use of autologous cancer cells as vaccines to augment anti-tumor immunity has been explored for some time (Oettgen et al., “The History of Cancer Immunotherapy”, In: Biologic Therapy of Cancer, Devita et al. (eds.) J. Lippincot Co., pp87-199, 1991). However, due to the weak immunogenicity exhibited by cancer cells, down regulation of MHC molecules, the lack of adequate costimulatory molecule expression and secretion of immunoinhibitory cytokines, the response to such vaccines has not resulted in long term efficacy. See, e.g., Armstrong T D and Jaffee E M, Surg Oncol Clin N Am. 11(3):681-96, 2002 and Bodey B et al., Anticancer Res 20(4):2665-76, 2000.

Numerous cytokines have been shown to play a role in regulation of the immune response to tumors. For example, U.S. Pat. No. 5,098,702 describes using combinations of TNF, IL-2 and IFN-beta in synergistically effective amounts to combat existing tumors and U.S. Pat. Nos. 5,078,996, 5,637,483 and 5,904,920 describe the use of GM-CSF for treatment of tumors. However, direct administration of cytokines for cancer therapy may not be practical, as they are often systemically toxic. (See, for example, Asher et al., J. Immunol. 146: 3227-3234, 1991 and Havell et al., J. Exp. Med. 167: 1067-1085, 1988.)

An expansion of this approach involves the use of genetically modified tumor cells which express cytokines, with activity demonstrated in tumor models using a variety of immunomodulatory cytokines, including IL-4, IL-2, TNF-alpha, G-CSF, IL-7, IL-6 and GM-CSF, as described in Golumbeck P T et al., Science 254:13-716, 1991; Gansbacher B et al., J. Exp. Med. 172:1217-1224, 1990; Fearon E R et al., Cell 60:397-403, 1990; Gansbacher B et al., Cancer Res. 50:7820-25, 1990; Teng M et al., PNAS 88:3535-3539, 1991; Columbo M P et al., J. Exp. Med. 174:1291-1298, 1991; Aoki et al., Proc Natl Acad Sci USA. 89(9):3850-4, 1992; Porgador A, et al., Nat Immun. 13(2-3):113-30, 1994; Dranoff G et al., PNAS 90:3539-3543, 1993; Lee C T et al., Human Gene Therapy 8:187-193, 1997; Nagai E et al., Cancer Immunol. Immonther. 47:2-80, 1998 and Chang A et al., Human Gene Therapy 11:839-850, 2000, respectively.

Clinical trials employing GM-CSF-expressing autologous or allogeneic cellular vaccines (GVAX) have commenced for treatment of prostate cancer, melanoma, lung cancer, pancreatic cancer, renal cancer, and multiple myeloma (Dummer R., Curr Opin Investig Drugs 2(6):844-8, 2001; Simons J et al., Cancer Res. 15;59(20):5160-8, 1999; Soiffer R et al., PNAS 95:13141-13146, 1998; Simons J et al., Cancer Res. 15; 57:1537-1546, 1997; Jaffee E et al., J. Clin Oncol. 19:145-156, 2001; and Salgia R et al., J. Clin Oncol. 21:624-630, 2003).

In yet another approach, autologous tumor cells were genetically altered to produce a costimulatory molecule, such as the B7-1 costimulatory molecule or allogeneic histocompatibility antigens (Salvadori et al. Hum. Gene Ther. 6:1299-1306, 1995 and Plaksin et al. Int. J. Cancer 59:796-801, 1994). While the use of genetically modified tumor cells has met with success in treatment of some forms of cancer, there remains a need for improved treatment regimens with greater efficacy and less side effects than therapies currently in use.

### SUMMARY OF THE INVENTION

The invention provides improved compositions and methods for the treatment of cancer in a mammal, typically a human, by administering a cytokine-expressing cellular vaccine combination comprising a cytokine-expressing cellular vaccine and at least one additional cancer therapeutic agent to a subject with cancer.

Administration of the a cytokine-expressing cellular vaccine combination results in enhanced therapeutic efficacy relative to administration of the cytokine-expressing cellular vaccine or the additional cancer therapeutic agent alone.

In one aspect of the invention, the cytokine expressing cellular vaccine expresses GM-CSF.

In another aspect of the invention, the cytokine-expressing cellular vaccine combination comprises cells that are autologous, allogeneic or bystander cells.

In a further aspect of the invention, a population of autologous, allogeneic or bystander cells are genetically modified to produce an effective amount of a cytokine, e.g. GM-CSF.

The at least one additional cancer therapeutic agent may be selected from the group consisting of an anti-CTLA4 antibody, an anti-4-1BB antibody, an anti-=CD40 antibody or CD40 ligand, interferon-alpha, IL-2, an anti-OX40 antibody or OX-40 ligand and a CpC oligonucleotide.

The same or a different population of autologous, allogeneic or bystander cells may be genetically modified to produce an effective amount of the at least one additional cancer therapeutic agent.

The autologous, allogeneic or bystander cells are rendered proliferation incompetent prior to administration to the subject.

In yet a further aspect of the invention, the at least one additional cancer therapeutic agent is administered as a chemical entity such as an antibody (e.g., an anti-CTLA4 antibody, an anti-4-1BB antibody, an anti-CD40 antibody and an anti-OX40 antibody) or as traditional chemotherapy, e.g., taxotere.

The cytokine-expressing cellular vaccine is typically administered subcutaneously or intratumorally. The at least one additional cancer therapeutic agent may be administered prior to, at eth same time as, or following administration of the cytokine-expressing cellular vaccine component of the combination.

The invention further provides compositions and kits comprising cytokine-expressing cellular vaccine combinations for use according to the description provided herein.

### BRIEF DESCRIPTION OF THE DRAWINGS

FIG. 1A is a schematic depiction of a standard combination study in B16F10 melanoma model.

FIG. 1B is a graphic depiction of the results of a study in C57B1/6 mice indicating tumor free survival of C57B1/6 mice following injection of a GM-CSF-secreting tumor cell vaccine in a prevention model. In the basic model, mice were vaccinated (sc) with 1×10⁶ irradiated tumor cells as indicated in the figure legend. Seven days later, mice were challenged (sc) with 1×10⁶ B16F10 tumor cells and followed for tumor development and survival.

FIG. 1C is a graphic depiction of the results of a study in C57B1/6 mice indicating tumor free survival of C57B1/6 mice following injection of a GM-CSF-secreting tumor cell vaccine in a treatment model. In the treatment model mice were challenged (sc) with 5×10⁴ B16F10 tumor cells and 3 days later treated with 1×10⁶ irradiated tumor vaccine as indicated in the figure legend. Mice were followed for tumor development and survival. AAV#81 is a B16F10 clone that was transduced with adenovirus expressing GM-CSF. AAV#81 produced an average of 500 ng GM-CSF/24 h/1×10⁶ cells.

FIGS. 2A-C illustrate the results of treatment with the combination of B16F10 or GM-CSF-secreting B16F10 (dB16gmtd) cells and an anti-CTLA-4 antibody in the B16F10 model. C57B16 mice were challenged with 1×10⁵ B16F10 tumor cells. One day later, mice were vaccinated with 3×10⁶ irradiated B16F10 or GM-CSF-secreting B16F10 (dB16gmtd) cells. One and three days after vaccination, mice were injected with 150 ug and 1000 ug of anti-CTLA-4 (9D9 or 9H10; FIG. 2A), respectively. Mice received a second vaccination of 3×10⁶ irradiated cells two days after the last anti-CTLA-4 treatment. Mice were then monitored twice weekly for the development of subcutaneous tumors. FIGS. 2B and 2C illustrate the combined effect of GM-CSF-secreting B16F10 (dB16gmtd) cells and 150 and 450 ug of anti-CTLA4 antibody for the 9D9 (FIG. 2B) and 9H10 (FIG. 2C), antibodies, respectively. dB16gmtd 150 ug and dB16gmtd 450 ug refer to 150 ug and 450 ug GM-CSF produced at the vaccine site, respectively. 150 ug and 100 ug of the 9D9 antibody was delivered on day 1 and 3 after the vaccine.

FIG. 3 is a schematic depiction of a study wherein the effect of GVAX was evaluated in combination with a 4-1BB monoclonal antibody.

FIGS. 4A-B illustrates the results of treatment with the combination of B16F10 or GM-CSF-secreting B16F10 (GVAX) cells and an anti-4-1BB antibody in the B16F10 model, wherein FIG. 4A illustrates the percentage of tumor-free mice and FIG. 4B illustrates percentage survival up to 80 days post challenge.

FIG. 5 graphically represents the study design wherein the effect of the combination of GVAX and CD40L was evaluated in a murine B16F10 model.

FIG. 6A illustrates the results of treatment with the combination of B16F10 and adenovirus-expressed CD40 ligand in the B16F10 model. On day 0, mice were challenged with 1×10⁵ live B16F10 mouse melanoma cells, and on day 3 after challenge, mice were vaccinated ventrally with 1×10⁶ B16-GM, followed by administration of further irradiated vaccine cells on day 3, day 4, day 5, and day 7, as shown in Table 3. Mice were monitored for tumor burden every 3-4 days for 3 months (n=10) and sacrificed on day 18, with spleens and blood collected for immunological assays.

FIGS. 6B and C illustrate the results of treatment with the combination of B16F1 and an anti-CD40 antibody in the B16F10 model. C57B16 mice were challenged dorsally with 1×10⁵ live B16F10 mouse melanoma cells. On day 3 after challenge mice were vaccinated ventrally with 1×10⁶ B16-GM. 100ug of anti CD40 antibody was administered per injection on DO and 2 or D2 and 4 (FIG. 6B) or D4 and 6; D7 and 9 or D10 and 12 (FIG. 6C). Mice were monitored for tumor burden every 3-4 days for 3 months (n=10) and sacrificed on day 18, with spleens and blood collected for immunological assays.

FIG. 7 graphically represents the study design wherein the effect of the combination of GVAX and interferon-alpha was evaluated in a murine B16F10 tumor model.

FIGS. 8A-B illustrate the results of treatment with the combination of B16F10 and an interferon-alpha in the B16F10 model. C57B16 mice were challenged with 1×10⁵ B16F10 tumor cells. Three days later, mice were vaccinated with 1×10⁶ irradiated B16F10 or GM-CSF-secreting B16F10 (B16.GM) cells. Four days after the vaccine IFN-alpha administration was initiated. IFN-alpha (2500 units/day; twice/week) was injected subcutaneously at the site of tumor challenge (FIG. 8A) the vaccination site (FIG. 8B). Mice were then monitored twice weekly for the development of subcutaneous tumors.

FIG. 9 graphically represents the study design wherein the effect of the combination of GVAX and IL-2 was evaluated in a murine B16F10 model.

FIG. 10 graphically represents the study design wherein the effect of the combination of GVAX and CpG ODN were evaluated in a murine B16F10 model.

### DETAILED DESCRIPTION

The present invention represents an improvement over cellular vaccines for the treatment of cancer that are currently in use in that the compositions and methods described herein comprise at least two components that act in concert to effect an improved therapeutic outcome for the patient under treatment.

### Definitions

The terms “regulating the immune response” or “modulating the immune response” as used herein refers to any alteration in a cell of the immune system or any alteration in the activity of a cell involved in the immune response. Such regulation or modulation includes an increase or decrease in the number of cells, an increase or decrease in the activity of the cells, or any other changes which can occur within the immune system. Cells involved in the immune response include, but are not limited to, T lymphocytes, B lymphocytes, natural killer (NK) cells, macrophages, eosinophils, mast cells, dendritic cells and neutrophils. In some cases, “regulating” or “modulating” the immune response means the immune response is stimulated or enhanced and in other cases “regulating” or “modulating” the immune response means suppression of the immune system. Stimulation of the immune system may include memory responses and/or future protection against subsequent antigen challenge.

The terms “cancer therapeutic agent”, “additional cancer therapeutic agent or treatment” and the like as used herein refer to any molecule or treatment that stimulates an anti-cancer response when used in combination with a cytokine-expressing cellular vaccine. In one aspect, the additional cancer therapeutic agent is expressed by a recombinant tumor cell and may be an immunomodulatory molecule”, i.e. a second cytokine. In another aspect, the additional cancer therapeutic agent is administered in the form of a protein or other chemical entity, e.g., an antibody or standard chemotherapeutic agent such as taxotere, provided in a pharmaceutically acceptable excipient. In yet another aspect, the cancer therapeutic agent is a standard treatment traditionally used in the treatment of cancer, e.g., radiation. In a further aspect, the “cancer therapeutic agent” is an agent or treatment, which is typically not considered in the treatment of cancer, but which when administered to a patient in combination with a cytokine-expressing cellular vaccine results in an improved therapeutic outcome for the patient under treatment.

The term “cytokine” or “cytokines” as used herein refers to the general class of biological molecules which effect/affect cells of the immune system. The definition is meant to include, but is not limited to, those biological molecules that act locally or may circulate in the blood, and which, when used in the compositions or methods of the present invention serve to regulate or modulate an individual's immune response to cancer. Exemplary cytokines for use in practicing the invention include but are not limited to IFN-alpha, IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, ICAM, macrophage colony stimulating factor (M-CSF), granulocyte colony stimulating factor (G-CSF) and granulocyte-macrophage colony stimulating factor (GM-CSF).

The term “cytokine-expressing cellular vaccine” as used herein refers to a composition comprising a population of cells that has been genetically modified to express a cytokine, e.g., GM-CSF, and that is administered to a patient as part of a cancer treatment regimen. The cells of such a “cytokine-expressing cellular vaccine” comprise a cytokine-encoding DNA sequence operably linked to expression and control elements such that the cytokine is expressed by the cells. The cells of the “cytokine-expressing cellular vaccine” are typically tumor cells and may be autologous or allogeneic to the patient undergoing treatment and or may be “bystander cells” that are mixed with tumor cells taken from the patient. A GM-CSF-expressing “cytokine-expressing cellular vaccine” may be referred to herein as “GVAX”.

The term “operably linked” as used herein relative to a recombinant DNA construct or vector means nucleotide components of the recombinant DNA construct or vector are directly linked to one another for operative control of a selected coding sequence. Generally, “operably linked” DNA sequences are contiguous, and, in the case of a secretory leader, contiguous and in reading frame, however, some sequences, e.g., enhancers do not have to be contiguous.

As used herein, the term “gene” or “coding sequence” means the nucleic acid sequence which is transcribed (DNA) and translated (mRNA) into a polypeptide in vitro or in vivo when operably linked to appropriate regulatory sequences. A “gene” typically comprises the coding sequence plus any non-coding sequences associated with the gene (e.g., regulatory sequences) and hence may or may not include regions preceding and following the coding region, e.g. 5′ untranslated (5′UTR) or “leader” sequences and 3′ UTR or “trailer” sequences, as well as intervening sequences (introns) between individual coding segments (exons). In contrast, a “coding sequence” does not include non-coding DNA.

The terms “gene-modified” and “genetically-modified” are used herein with reference to a cell or population of cells wherein a nucleic acid sequence has been introduced into the cell or population of cells. The nucleic acid sequence may be heterologous to the cell(s), or it may be an additional copy or improved version of a nucleic acid sequence already present in the cell(s). The cell(s) may be genetically-modified by physical or chemical methods or by the use of recombinant viruses. Chemical and physical methods such as calcium phosphate, electroporation and pressure mediated transfer of genetic material into cells are often used. Several recombinant viral vectors which find utility in effective delivery of genes into mammalian cells include, for example, retroviral vectors, adenovirus vectors, adenovirus-associated vectors (AAV), herpes virus vectors, pox virus vectors. In addition, non-viral means of introduction, for example, naked DNA delivered via liposomes, receptor-mediated delivery, calcium phosphate transfection, electroporation, particle bombardment (gene gun), or pressure-mediated delivery may also be employed to introduce a nucleic acid sequence into a cell or population of cells to render them “gene-modified” or “genetically-modified.

As used herein, the terms “tumor” and “cancer” refer to a cell that exhibits a loss of growth control and forms unusually large clones of cells. Tumor or cancer cells generally have lost contact inhibition and may be invasive and/or have the ability to metastasize.

The term “antigen from a tumor cell” and “tumor antigen” and “tumor cell antigen” may be used interchangeably herein and refer to any protein, carbohydrate or other component derived from or expressed by a tumor cell which is capable of eliciting an immune response. The definition is meant to include, but is not limited to, whole tumor cells with all of the tumor-associated antigens, tumor cell fragments, plasma membranes taken from a tumor cell, proteins purified from the cell surface or membrane of a tumor cell, or unique carbohydrate moieties associated with the cell surface of a tumor cell. The definition also includes those antigens from the surface of the cell which require special treatment of the cells to access.

The term “systemic immune response” as used herein means an immune response which is not localized, but affects the individual as a whole.

The term “gene therapy” as used herein means the treatment or prevention of cancer by means of ex vivo or in vivo delivery, through viral or non-viral vectors, of compositions containing a recombinant genetic material.

The term “ex vivo” delivery as used herein means the introduction, outside of the body of a human, of compositions containing a genetic material into a cell, tissue, organoid, organ, or the like, followed by the administration of cell, tissue, organoid, organ, or the like which contains such introduced compositions into the body of the same (autologous) or a different (allogeneic) human, without limitation as to the formulation, site or route of administration.

The terms “inactivated cells”, “non-dividing cells” and “non-replicating cells” may be used interchangeably herein and refer to cells that have been treated rendering them proliferation incompetent, e.g., by irradiation. Such treatment results in cells which are unable to undergo mitosis, but still retain the capability to express proteins such as cytokines or other cancer therapeutic agents. Typically a minimum dose of about 3500 rads is sufficient, although doses up to about 30,000 rads are acceptable. Effective doses include, but are not limited to 5000 to 10000 rads. Numerous methods of inactivating cells, such as treatment with Mitomycin C, are known in the art. Any method of inactivation which renders cells incapable of cell division, but allows the cells to retain the ability to express proteins is included within the scope of the present invention.

As used herein “treatment” of an individual or a cell is any type of intervention used in an attempt to alter the natural course of the individual or cell. Treatment includes, but is not limited to, administration of e.g., a cytokine-expressing cellular vaccine and at least one additional cancer therapeutic agent or treatment, and may be performed either prophylactically or subsequent to diagnosis as part of a primary or follow-up therapeutic regimen.

The term “administering” as used herein refers to the physical introduction of a composition comprising a cytokine-expressing cellular vaccine and at least one additional cancer therapeutic agent or treatment to a patient with cancer. Any and all methods of introduction are contemplated according to the invention, the method is not dependent on any particular means of introduction and is not to be so construed. Means of introduction are well-known to those skilled in the art, and also are exemplified herein.

The term “co-administering” as used herein means a process whereby the combination of a cytokine-expressing cellular vaccine and at least one additional cancer therapeutic agent or treatment is administered to the same patient. The cytokine-expressing cellular vaccine and additional cancer therapeutic may be administered simultaneously, at essentially the same time, or sequentially. If administration-takes place sequentially, the cytokine-expressing cellular vaccine may be administered before or after a given additional cancer therapeutic agent or treatment. The cytokine-expressing cellular vaccine and additional cancer therapeutic agent or treatment need not be administered by means of the same vehicle, the cellular vaccine and the additional agent or treatment may be administered one or more times and the number of administrations of each component of the combination may be the same or different.

The term “therapeutically effective amount” or “therapeutically effective combination” as used herein refers to an amount or dose of a cytokine-expressing cellular vaccine together with the amount or dose of an additional agent or treatment that is sufficient to modulate, either by stimulation or suppression, the systemic immune response of an individual. The amount of cytokine-expressing cellular vaccine in a given therapeutically effective combination may be different for different individuals, different tumor types and will be dependent upon the one or more additional agents or treatments included in the combination. The “therapeutically effective amount” is determined using procedures routinely employed by those of skill in the art such that an “improved therapeutic outcome” results.

As used herein, the terms “improved therapeutic outcome” and “enhanced therapeutic efficacy”, relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including an increase in life expectancy or an improvement in quality of life.

The term “reversal of an established tumor” as used herein means the suppression, regression, partial or complete disappearance of a pre-existing tumor. The definition is meant to include any diminution in the size, growth rate, appearance or cellular compositions of a preexisting tumor.

The terms “individual”, “subject” as referred to herein is a vertebrate, preferably a mammal, and typically refers to a human.

### General Techniques

The practice of the present invention will employ, unless otherwise indicated, conventional techniques of molecular biology, microbiology, cell biology, biochemistry and immunology, which are within the knowledge of those of skill of the art. Such techniques are explained fully in the literature, such as, “Molecular Cloning: A Laboratory Manual”, second edition (Sambrook et al., 1989); “Current Protocols in Molecular Biology” (F. M. Ausubel et al., eds., 1987); “Animal Cell Culture” (R. I. Freshney, ed., 1987), each of which is hereby expressly incorporated herein by reference.

### Cancer Targets

The methods and compositions of the invention provide an improved therapeutic approach to the treatment of cancer by co-administration of a cytokine-expressing cellular vaccine and at least one additional cancer therapeutic agent or treatment to a patient with cancer. “Cancer” as used herein includes cancer localized in tumors, as well as cancer not localized in tumors, such as, for instance, cancer cells that expand from a local tumor by invasion (i.e., metastasis). The invention finds utility in the treatment of any form of cancer, including, but not limited to, cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

### Introduction of Cytokine and Cancer Therapeutic Agent-Encoding Nucleic Acid Sequences into Cells

In one aspect of the invention, a nucleic acid sequence (i.e., a recombinant DNA construct or vector) encoding a cytokine operably linked to a first promoter, alone or in combination with a nucleic acid sequence encoding a cancer therapeutic agent operably linked to a second promoter is introduced into a cell or population of cells. Any and all methods of introduction into a cell or population of cells, typically tumor cells, are contemplated according to the invention, the method is not dependent on any particular means of introduction and is not to be so construed. The cytokine-encoding nucleic acid sequence may be introduced into the same or a different population of cells as the cancer therapeutic agent-encoding nucleic acid sequence.

The “vector” may be a DNA molecule such as a plasmid, virus or other vehicle, which contains one or more heterologous or recombinant DNA sequences, e.g., a nucleic acid sequence encoding a cytokine or additional cancer therapeutic agent under the control of a functional promoter and possibly also an enhancer that is capable of functioning as a vector, as understood by those of ordinary skill in the art. An appropriate viral vector includes, but is not limited to, a retrovirus, a lentivirus, an adenovirus (AV), an adeno-associated virus (AAV), a simian virus 40 (SV-40), a bovine papilloma virus, an Epstein-Barr virus, a herpes virus, a vaccinia virus, a Moloney murine leukemia virus, a Harvey murine sarcoma virus, a murine mammary tumor virus, and a Rous sarcoma virus.

Any suitable vector can be employed that is appropriate for introduction of nucleic acids into eukaryotic tumor cells, or more particularly animal tumor cells, such as mammalian, e.g., human, tumor cells. Preferably the vector is compatible with the tumor cell, e.g., is capable of imparting expression of the coding sequence for a cytokine or cancer therapeutic agent, and is stably maintained or relatively stably maintained in the tumor cell. Desirably the vector comprises an origin of replication and the vector may or may not also comprise a “marker” or “selectable marker” function by which the vector can be identified and selected. While any selectable marker can be used, selectable markers for use in such expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern and Berg, J., 1982), mycophenolic acid (Mulligan and Berg, 1980), puromycin, zeomycin, hygromycin (Sugden et al., 1985) or G418.

In practicing the methods of the present invention, a vector comprising a nucleic acid sequence encoding a cytokine or additional cancer therapeutic agent may be transferred to a cell in vitro, preferably a tumor cell, using any of a number of methods which include but are not limited to electroporation, membrane fusion with liposomes, Lipofectamine treatment, high velocity bombardment with DNA-coated microprojectiles, incubation with calcium phosphate-DNA precipitate, DEAE-dextran mediated transfection, infection with modified viral nucleic acids, direct microinjection into single cells, etc. Procedures for the cloning and expression of modified forms of a native protein using recombinant DNA technology are generally known in the art, as described in Ausubel, et al., 1992 and Sambrook, et al, 1989, expressly incorporated by reference, herein.

Reference to a vector or other DNA sequences as “recombinant” merely acknowledges the operable linkage of DNA sequences which are not typically operably linked as isolated from or found in nature. A “promoter” is a DNA sequence that directs the binding of RNA polymerase and thereby promotes RNA synthesis. Enhancers” are cis-acting elements that stimulate or inhibit transcription of adjacent genes. An enhancer that inhibits transcription also is termed a “silencer”. Enhancers can function (i.e. be operably linked to a coding sequence) in either orientation, over distances of up to several kilobase pairs (kb) from the coding sequence and from a position downstream of a transcribed region. Regulatory (expression/control) sequences are operatively linked to a nucleic acid coding sequence when the expression/control sequences regulate the transcription and, as appropriate, translation of the nucleic acid sequence. Thus expression/control sequences can include promoters, enhancers, transcription terminators, a start codon (i.e., ATG) in front of the coding sequined, splicing signal for introns and stop codons.

Recombinant vectors for the production of cellular vaccines of the invention provide all proper transcription, translation and processing signals (e.g., splicing and polyadenylation signals) such that the coding sequence for the cytokine or cancer therapeutic agent are appropriately transcribed and translated in the tumor cells into which they are introduced. The manipulation of such signals to ensure appropriate expression in host cells is within the skill of the ordinary skilled artisan. The coding sequence for the cytokine and cancer therapeutic agent may be under control of (i.e., operably linked to) their own native promoter, or a non-native (i.e. heterologous) promoter, including a constitutive promoter, such as the cytomegalovirus (CMV) immediate early promoter/enhancer, the Rous sarcoma virus long terminal repeat (RSV-LTR) or the SV-40 promoter.

Alternately, a tissue-specific promoter (a promoter that is preferentially activated in a particular type of tissue and results in expression of a gene product in that tissue) can be used in the vector. Such promoters include but are not limited to a liver specific promoter (Ill C R, et al., Blood Coagul Fibrinolysis 8 Suppl 2:S23-30, 1997) and the EF-1 alpha promoter (Kim D W et al. Gene. 91(2):217-23, 1990, Guo Z S et al. Gene Ther. 3(9):802-10, 1996; U.S. Pat. Nos. 5,266,491 and 5,225,348, each of which expressly incorporated by reference herein). Inducible promoters also find utility in practicing the methods described herein, such as a promoter containing the tet responsive element (TRE) in the tet-on or tet-off system as described (ClonTech and BASF), the metallothienein promoter which can be upregulated by addition of certain metal salts and rapamycin inducible promoters (Rivera et al., 1996, Nature Med, 2(9):1028-1032; Ye et al., 2000, Science 283: 88-91; Sawyer T K et al., 2002, Mini Rev Med Chem. 2(5):475-88). Large numbers of suitable tissue-specific or regulatable vectors and promoters for use in practicing the current invention are known to those of skill in the art and many are commercially available.

Exemplary vector systems for use in practicing the invention include the retroviral MFG vector, described in U.S. Pat. No. 5,637,483, expressly incorporated by reference herein. Other useful retroviral vectors include pLJ, pEm and [alpha]SGC, described in U.S. Pat. No. 5,637,483 (in particular Example 12), U.S. Pat. Nos. 6,506,604, 5,955,331 and U.S. Ser. No. 09/612,808, each of which is expressly incorporated by reference herein.

Further exemplary vector systems for use in practicing the invention include second, third and fourth generation lentiviral vectors, U.S. Pat. Nos. 6,428,953, 5,665,577 and 5,981,276 and WO 00/72686, each of which is expressly incorporated by reference herein.

Further exemplary vector systems for use in practicing the present invention include adenoviral vectors, described for example in U.S. Pat. No. 5,872,005 and WO 00/72686, each of which is expressly incorporated by reference herein.

Yet another vector system that is preferred in practicing the methods described herein is a recombinant adeno-associated vector (rAAV) system, described for example in WO98/46728, WO 00/72686, Samulski et al., Virol. 63:3822-3828 (1989) and U.S. Pat. Nos. 5,436,146, 5,753,500, 6037177, 6040183 and 6093570, each of which is expressly incorporated by reference herein.

### Cytokines

Cytokines and combinations of cytokines have been shown to play an important role in the stimulation of the immune system. The term “cytokine” is understood by those of skill in the art, as referring to any immunopotentiating protein (including a modified protein such as a glycoprotein) that enhances or modifies the immune response to a tumor present in the host. The cytokine typically enhances or modifies the immune response by activating or enhancing the activity of cells of the immune system and is not itself immunogenic to the host.

It follows from the results presented herein that a variety of cytokines will find use in the present invention. Exemplary cytokines for use in practicing the invention include but are not limited to IFN-alpha, IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, macrophage colony stimulating factor (M-CSF), granulocyte colony stimulating factor (G-CSF) and granulocyte-macrophage colony stimulating factor (GM-CSF). The cytokine may be from any source, however, optimally the cytokine is of murine or human origin (a native human or murine cytokine) or is a sequence variant of such a cytokine, so long as the cytokine has a sequence with substantial homology to the human form of the cytokine and exhibits a similar activity on the immune system. It follow that cytokines with substantial homology to the human forms of IFN-alpha, IFN-beta, and IFN-gamma, IL-1 to IL-29, TNF-alpha, TNF-beta, EPO, MIP3a, ICAM, M-CSF, G-CSF and GM-CSF are useful in practicing the invention, so long as the homologous form exhibits the same or a similar effect on the immune system. Proteins that are substantially similar to any particular cytokine, but have relatively minor changes in protein sequence find use in the present invention. It is well known that small alterations in protein sequence may not disturb the functional activity of a protein molecule, and thus proteins can be made that function as cytokines in the present invention but differ slightly from current known or native sequences.

### Variant Sequences

Homologues and variants of native human or murine cytokines and additional cancer therapeutic agent are included within the scope of the invention. As used herein, the term “sequence identity” means nucleic acid or amino acid sequence identity between two or more aligned sequences and is typically expressed as a percentage (“%”). The term “% homology” is used interchangeably herein with the term “% identity” or “% sequence identity” and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence typically has greater than 80% sequence identity over a length of the given sequence. Preferred levels of sequence identity include, but are not limited to, 80, 85, 88, 89, 90, 91, 92, 93, 94, 95, 96, 97, 98 or 99% or more sequence identity to a native cytokine or cancer therapeutic agent amino acid or nucleic acid sequence, as described herein.

Exemplary computer programs that can be used to determine the degree of identity between two sequences include, but are not limited to, the suite of BLAST programs, e.g., BLASTN, BLASTX, TBLASTX, BLASTP and TBLASTN, all of which are publicly available on the Internet. See, also, Altschul, S. F. et al. Mol. Biol. 215:403-410, 1990 and Altschul, S. F. et al. Nucleic Acids Res. 25:3389-3402, 1997, expressly incorporated by reference herein. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. In determining sequence identity, both BLASTN and BLASTX (i.e. version 2.2.5) are run using default parameters of an open gap penalty of 11.0, and an extended gap penalty of 1.0, and utilize the BLOSUM-62 matrix. [See, Altschul, et al., 1997, supra.] A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about TM-5° C. (5° below the Tm of the probe]) “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe, while high stringency conditions are used to identify sequences having about 80% or more sequence identity with the probe.

Moderate and high stringency hybridization conditions are well known in the art (see, for example, Sambrook, et al, 1989, Chapters 9 and [11,] and in Ausubel, F. M., et al., 1993, expressly incorporated by reference herein). An example of high stringency conditions includes hybridization at about 42° C. in 50% formamide, 5×SSC, 5× Denhardt's solution, 0.5% SDS and 100 mg/ml denatured carrier DNA followed by washing two times in 2×SSC and 0.5% SDS at room temperature and two additional times in 0.1×SSC and 0.5% SDS at 42° C.

### One or More Additional Agents or Treatments

As detailed herein, the present invention is directed to a method of improving an individual's immune response to cancer (e.g., a target cancer antigen or antigens) by co-administering a cytokine-expressing cellular vaccine (e.g., GM-CSF; GVAX) and at least one additional cancer therapeutic agent or treatment to a patient with cancer. Cancer therapeutic agents or treatments for use in practicing the invention include, but are not limited to, adhesion or accessory molecules, other biological response modifiers, chemotherapeutic agents, radiation treatment and combinations thereof.

### Cancer Therapeutic Agents

Embodiments of the present invention include the administration of the combination of a cytokine-expressing cellular vaccine and at least one additional cancer therapeutic agent. Cancer therapeutic agents for use in practicing the invention include those listed in Tables 1A and 1B. These include agents from each of the major classes of cancer therapeutic agents, including but not limited to: alkylating agents, alkaloids, antimetabolites, anti-tumor antibiotics, nitrosoureas, hormonal agonists/antagonists and analogs, immunomodulators, photosensitizers, enzymes and others. In some embodiments, the cancer therapeutic agent is a cytokine or co-stimulatory molecule. In other embodiments the cancer therapeutic agent is for example CpG (a CG dinucleotide that mimics bacterial DNA; a HSP (heat shock protein); a Cox-2 inhibitor; an anti-41BB mAb; IL-2, IL-18; an anti-CTLA-4 monoclonal antibody; an anti-CD40 or membrane/soluble CD40 ligand; interferon alpha (IFN-alpha); Iressa; anti-epidermal growth factor R (EGF-R); or anti-VEGF R. Preferred cancer therapeutic agents include, but are not limited to, 5-fluorouracil, cisplatin, doxorubicin, estramustine, etoposide, mitoxantrone, docetaxel (TAXOTERE™), paclitaxel (TAXOL™), and retro-gutless adeno, IL-15, chemo-taxotere and erastomustin (plus steroids).

TABLE 1A
CANCER THERAPEUTIC AGENTS

| | ALKYLATING | ANTBIOTICS | | | |
|---|---|---|---|---|---|
| ALKALOIDS | AGENTS | AND ANALOGS | ANTIMETABOLITES | ENZYMES | IMMUNOMODULATORS |
| Docetaxel | Alkyl Sulfonates | Aclacinomycins | Folic Acid Analogs | L-Asparaginase | Interferon-α |
| (TAXOTERE ™) | | | | | |
| Etoposide | Busulfan | Actinomycin F₁ | Denopterin | Pegasargase | Interferon-β |
| Irinotecan | Improsulfan | Anthramycin | Edatrexate | | Interferon-γ |
| Paclitaxel | Piposulfan | Azaserine | Methotrexate | | Interferon-alpha-2a |
| (TAXOL ™) | | | | | |
| Teniposide | | Bleomycins | Piritrexim | | Interleukin-2 |
| Topotecan | Aziridines | Cactinomycin | Pteropterin | | Lentinan |
| Vinblastine | Benzodepa | Carubicin | Tomudex ® | | Propagermanium |
| Vincristine | Carboquone | Carzinophilin | Trimetrexate | | PSK |
| Vendesine | Meturedepa | Chromomycins | | | Roquinimex |
| Vinorelbine | Uredepa | Dactinomycin | Purine Analogs | | Rituximab |
| | | Daunorubicin | Cladribine | | Sizofiran |
| | Ethylenimines and | 6-Diazo-5-oxo- | Fludarabine | | Trastuzumab |
| | Methylmelamines | L-norleucine | | | |
| | Altretamine | Doxorubicin | 6-Mercaptopurine | | Ubenimex |
| | Triethylenemelamine | Epirubicin | Thiamiprine | | Cyclophosphamide/ |
| | | | | | cytoxan |
| | Triethylenephosphoramide | Idarubicin | Thioguanine | | |
| | Triethylenethiophosphoramide | Menogaril | | | |
| | | Mitomycins | | | |
| | | Mitoxantrone | Pyrimidine Analogs | | |
| | Nitrogen Mustards | Mycophenolic | Ancitabine | | |
| | | Acid | | | |
| | Chlorambucil | Nogalamycin | 5-Azacytidine | | |
| | Chlomaphazine | Olivomycins | 6-Azauridine | | |
| | Cyclophosphamide | Peplomycin | Carmofur | | |
| | Estramustine | Pirarubicin | Cytarabine | | |
| | Ifosfamide | Plicamycin, | Doxifluridine | | |
| | Mechlorethamine | Porfiromycin | Emitefur | | |
| | Mechlorethamine Oxide | Puromycin | Enocitabine | | |
| | Hydrochloride | | | | |
| | Melphalan | Streptonigrin | Floxuridine | | |
| | Novembichin | Streptozocin | Fluorouracil | | |
| | | Valrubicin | | | |
| | Perfosfamide | Tubercidin | Gemcitabine | | |
| | Phenesterine | Zinostatin | Tegafur | | |
| | Prednimustine | Zorubicin | | | |
| | Trofosfamide | | | | |
| | Uracil Mustard | | | | |
| | Carboplatin | | | | |
| | Cisplatin | | | | |
| | Miboplatin | | | | |
| | Oxaliplatin | | | | |
| | Others | | | | |
| | Dacarbazine | | | | |
| | Mannomustine | | | | |
| | Mitobronitol | | | | |
| | Mitolactol | | | | |
| | Thiotepa | | | | |
| | Pipobroman | | | | |
| | Temozolomide | | | | |

TABLE 1B
CANCER THERAPEUTIC AGENTS

| | | HORMONE | |
|---|---|---|---|
| | | ANTAGONISTS/ | |
| | | AGONISTS & | |
| NITROSOUREAS | OTHERS | ANALOGS | PHOTOSENSITIZER |
| Carmustine | Aceglatone | Dexamethasone | Porfimer Sodium |
| Chlorozotocin | Amsacrine | Prednisone | |
| Fotemustine | Bisantrene | | |
| Lomustine | Defosfamide | Androgens | |
| Nimustine | Demecolcine | Calusterone | |
| Ranimustine | Diaziquone | Dromostanolone | |
| | Eflornithine | Epitiostanol | |
| | Elliptinium Acetate | Mepitiostane | |
| | Etoglucid | Testolactone | |
| | Fenretinide | | |
| | Finasteride | Antiadrenals | |
| | Gallium Nitrate | Aminoglutethimide | |
| | Hydroxyurea | Mitotane | |
| | Lonidamine | Trilostane | |
| | Miltefosine | | |
| | Mitoguazone | Antiandrogens | |
| | Mopidamol | Bicalutamide | |
| | Nitracrine | Flutamide | |
| | Pentostatin | Nilutamide | |
| | Phenamet | | |
| | Podophyllinic Acid 2- | Antiestrogens | |
| | Ethylhydrazide | | |
| | Procarbazine | Droloxifene | |
| | Razoxane | Tamoxifen | |
| | Sobuzoxane | Toremifene | |
| | Spirogermanium | Exemestane | |
| | Amsacrine | Aromatase Inhibitors | |
| | Tretinoin | Aminoglutethimide | |
| | Tenuazonic Acid | Anastrozole | |
| | Triaziquone | Fadrozole | |
| | 2,2′,2″- | Formestane | |
| | Triclorotriethylamine, | | |
| | Urethan | Letrozole | |
| | Topotecan | | |
| | | Estrogens | |
| | | Fosfestrol | |
| | | Hexestrol | |
| | | Polyestradiol Phosphate | |
| | | LHRH Analogs | |
| | | Buserelin | |
| | | Goserelin | |
| | | Leuprolide | |
| | | Triptorelin, | |
| | | Progestogens | |
| | | Chlormadinone Acetate | |
| | | Medroxyprogesterone | |
| | | Megestrol Acetate | |
| | | Melengestrol | |

### Cellular Vaccine Combinations

Granulocyte-macrophage colony stimulating factor (GM-CSF) is a cytokine produced by fibroblasts, endothelial cells, T cells and macrophages. This cytokine has been shown to induce the growth of hematopoetic cells of granulocyte and macrophage lineages. In addition, it also activates the antigen processing and presenting function of dendritic cells, which are the major antigen presenting cells (APC) of the immune system. Results from animal model experiments have convincingly shown that GM-CSF producing tumor cells (i.e. GVAX) are able to induce an immune response against parental, non-transduced tumor cells.

Autologous and allogeneic cancer cells that have been genetically modified to express a cytokine, e.g., GM-CSF, followed by readministration to a patient for the treatment of cancer are described in U.S. Pat. Nos. 5,637,483, 5,904,920 and 6,350,445, expressly incorporated by reference herein. A form of GM-CSF-expressing genetically modified cancer cells or a “cytokine-expressing cellular vaccine” (also known as “GVAX”), for the treatment of pancreatic cancer is described in U.S. Pat. Nos. 6,033,674 and 5,985,290, expressly incorporated by reference herein. A universal immunomodulatory cytokine-expressing bystander cell line is described in U.S. Pat. No. 6,464,973, expressly incorporated by reference herein.

Clinical trials employing GM-CSF-expressing autologous or allogeneic cellular vaccines (GVAX) have been undertaken for treatment of prostate cancer, melanoma, lung cancer, pancreatic cancer, renal cancer, and multiple myeloma, however, the question still remains whether the immune responseto GM-CSF alone will be powerful enough to slow or eradicate large or fast growing malignancies.

The present invention provides a method of stimulating an improved immune response to cancer in a mammalian, preferably a human, patient. Desirably, the method effects a systemic immune response, i.e., a T-cell response and/or a B-cell response, to the cancer. The method comprises administering to the patient a cytokine-expressing cellular vaccine and at least one additional cancer therapeutic agent or treatment, wherein the cellular vaccine comprises cells which express a cancer antigen, the cancer antigen is an antigen of the cancer in the patient and the composition is rendered proliferation incompetent, such as by irradiation. Upon administration of the composition, an immune response to the cancer is stimulated. In one approach, the cytokine-expressing cellular vaccine combination comprises a single population of cells that is modified to express a cytokine and at least one additional cancer therapeutic agent. In another approach, the vaccine comprises two or more populations of cells individually modified to express each component of the vaccine. In yet another approach, the cytokine-expressing cellular vaccine combination comprises a population of cells that is modified to express a cytokine together which is administered in combination with at least one additional cancer therapeutic agent or treatment.

In general, a cytokine-expressing cellular vaccine combination for use in practicing the invention comprises tumor cells selected from the group consisting of autologous tumor cells, allogeneic tumor cells and tumor cell lines (i.e., bystander cells).

In other embodiments, the cells of the cytokine-expressing cellular vaccine combination are cryopreserved prior to administration. In a preferred embodiment, the cells of the cytokine-expressing cellular vaccine combination are administered to the same individual from whom they were derived. In another preferred embodiment, the cells of the cytokine-expressing cellular vaccine combination and the tumor are derived from different individuals. In certain preferred embodiments, the type of tumor being treated is selected from the group consisting of cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

In one aspect of the invention, the cells of the cytokine-expressing cellular vaccine combination comprise gene-modified cells that are of one type for the expression of the cytokine and of another different type for expression of the one or more additional cancer therapeutic agents. By way of example, in one approach, the cytokine-expressing cellular vaccine (i.e., GVAX) may be provided as an allogeneic or bystander cell line while the one or more additional cancer therapeutic agents may be expressed by autologous cells. In another approach, the one or more additional cancer therapeutic agents may be expressed by an allogeneic or bystander cell line while the cytokine (i.e., GM-CSF) is expressed by autologous cells.

Direct comparison of murine tumor cells transduced with various cytokines demonstrated that GM-CSF-secreting tumor cells induced the best overall anti-tumor protection. In one preferred embodiment, the cytokine expressed by the cytokine-expressing cellular vaccine of the invention is GM-CSF (generally referred to herein as “GVAX”). The preferred coding sequence for GM-CSF is the genomic sequence described in Huebner K. et al., Science 230(4731):1282-5,1985, however, in some cases the cDNA form of GM-CSF finds utility in practicing the invention (Cantrell et al., Proc. Natl. Acad. Sci., 82, 6250-6254, 1985).

Preferably, the cytokine-expressing cellular vaccine combination is irradiated at a dose of from about 50 to about 200 rads/min, even more preferably, from about 120 to about 140 rads/min prior to administration to the patient. Preferably, the cells are irradiated with a total dose sufficient to inhibit substantially 100% of the cells, from further proliferation. Thus, desirably the cells are irradiated with a total dose of from about 10,000 to 20,000 rads, optimally, with about 15,000 rads.

### Autologous

The use of autologous cytokine-expressing cells in the vaccines of the invention provides advantages since each patient's tumor expresses a unique set of tumor antigens that can differ from those found on histologically-similar, MHC-matched tumor cells from another patient. See, e.g., Kawakami et al., J. Immunol., 148, 638-643 (1992); Darrow et al., J. Immunol., 142, 3329-3335 (1989); and Hom et al., J. Immunother., 10, 153-164 (1991). In contrast, MHC-matched tumor cells provide the advantage that the patient need not be taken to surgery to obtain a sample of their tumor for vaccine production.

In one preferred aspect, the present invention comprises a method of treating cancer by carrying out the steps of: (a) obtaining tumor cells from a mammal harboring a tumor; (b) modifying the tumor cells to render them capable of producing an increased level of a cytokine and at least one additional cancer therapeutic agent relative to unmodified tumor cells; (c) rendering the modified tumor cells proliferation incompetent; and (d) readministering the modified tumor cells to the mammal from which the tumor cells were obtained or to a mammal with the same MHC type as the mammal from which the tumor cells were obtained. The administered tumor cells are autologous and MHC-matched to the host.

The same autologous tumor cells may express both the cytokine and cancer therapeutic agent(s) or each may be expressed by a different autologous tumor cell. In one aspect of the invention, an autologous tumor cell is modified by introduction of a vector comprising a nucleic acid sequence encoding a cytokine, operably linked to a promoter and expression/control sequences necessary for expression thereof. In another aspect, the same autologus tumor cell or a second autologous tumor cell is modified by introduction of a vector comprising a nucleic acid sequence encoding at least one additional cancer therapeutic agent operably linked to a promoter and expression/control sequences necessary for expression thereof. The nucleic acid sequence encoding the cytokine and additional cancer therapeutic agent(s) may be introduced into the same or a different autologous tumor cell using the same or a different vector. The nucleic acid sequence encoding the cytokine or cancer therapeutic agent may or may not further comprise a selectable marker sequence operably linked to a promoter.

### Allogeneic

Researchers have sought alternatives to autologous and MHC-matched cells as tumor vaccines, as reviewed by Jaffee et al., Seminars in Oncology, 22, 81-91 (1995). Early tumor vaccine strategies were based on the understanding that the vaccinating tumor cells function as the antigen presenting cells (APCs) that present tumor antigens on their MHC class I and II molecules, and directly activate the T cell arm of the immune system. The results of Huang et al. (Science, 264, 961-965, 1994), indicate that professional APCs of the host rather than the vaccinating tumor cells prime the T cell arm of the immune system by secreting cytokine(s) such as GM-CSF such that bone marrow-derived APCs are recruited to the region of the tumor. The bone marrow-derived APCs take up the whole cellular protein of the tumor for processing, and then present the antigenic peptide(s) on their MHC class I and II molecules, thereby priming both the CD4+ and the CD8+ T cell arms of the immune system, resulting in a systemic tumor-specific anti-tumor immune response. These results suggest that it may not be necessary or optimal to use autologous or MHC-matched tumor cells in order to elicit an anti-cancer immune response and that the transfer of allogeneic MHC genes (from a genetically dissimilar individual of the same species) can enhance tumor immunogenicity. More specifically, in certain cases, the rejection of tumors expressing allogeneic MHC class I molecules resulted in enhanced systemic immune responses against subsequent challenge with the unmodified parental tumor, as reviewed in Jaffee et al., supra, and Huang et al., supra.

As described herein, a “tumor cell line” comprises cells that were initially derived from a tumor. Such cells typically are transformed (i.e., exhibit indefinite growth in culture).

In one preferred aspect, the invention provides a method for treating cancer by carrying out the steps of: (a) obtaining a tumor cell line; (b) modifying the tumor cell line to render the cells capable of producing an increased level of a cytokine alone or in combination with at least one additional cancer therapeutic agent relative to the unmodified tumor cell line; (c) rendering the modified tumor cell line prolifereastion incompetent; and (d) administering the tumor cell line to a mammalian host having at least one tumor that is the same type of tumor as that from which the tumor cell line was obtained. The administered tumor cell line is allogeneic and is not MHC-matched to the host. Such allogeneic lines provide the advantage that they can be prepared in advance, characterized, aliquoted in vials containing known numbers of cytokine-expressing cells and stored such that well characterize cells are available for administration to the patient. Methods for the production of gene-modified allogeneic cells are described for example in WO 00/72686A1, expressly incorporated by reference herein.

In one approach to preparing a cytokine-expressing cellular vaccine comprising gene-modified allogeneic cells, a cytokine and cancer therapeutic agent-encoding nucleic acid sequence is introduced into a cell line that is an allogeneic tumor cell line (i.e., derived from an individual other than the individual being treated). In another approach, a cytokine and cancer therapeutic agent encoding nucleic acid sequence are introduced into separate allogeneic tumor cell lines. The cell or population of cells may be from a tumor cell line of the same type as the tumor or cancer being treated. The tumor and/or tumor cell line may be of any form of cancer, including, but not limited to, carcinoma of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; or another tumor, including a melanoma, teratocarcinoma, neuroblastoma, glioma, adenocarcinoma and non-small lung cell carcinoma.

In one aspect of the invention, the allogeneic tumor cell is modified by introduction of a vector comprising a nucleic acid sequence encoding a cytokine, operably linked to a promoter and expression control sequences necessary for expression thereof. In another aspect, the same allogeneic tumor cell or a second allogeneic tumor cell is modified by introduction of a vector comprising a nucleic acid sequence encoding at least one additional cancer therapeutic agent operably linked to a promoter and expression control sequences necessary for expression thereof. The nucleic acid sequence encoding the cytokine and additional cancer therapeutic agent(s) may be introduced into the same or a different allogeneic tumor cell using the same or a different vector. The nucleic acid sequence encoding the cytokine or cancer therapeutic agent may or may not further comprise a selectable marker sequence operably linked to a promoter.

Desirably, the allogeneic cell line expresses high levels of the cytokine, e.g., GM-CSF. Preferably, the universal bystander cell line expresses at least about 500 ng GM-CSF/10 cells/24 hours

In practicing the invention, one or more allogeneic cell lines are incubated with an autologous cancer antigen, e.g., an autologous tumor cell (which together comprise an allogeneic cell line composition), then the allogeneic cell line composition is administered to the patient. Typically, the cancer antigen is provided by (on) a cell of the cancer to be treated, i.e., an autologous cancer cell. In such cases, the composition is rendered proliferation-incompetent by irradiation, wherein the allogeneic cells and cancer cells are plated in a tissue culture plate and irradiated at room temperature using a Cs source, as detailed above. The ratio of allogeneic cells to autologous cancer cells in a given administration will vary dependent upon the combination.

Any suitable route of administration can be used to introduce an allogeneic cell line composition into the patient, preferably, the composition is administered subcutaneously or intratumorally.

The use of allogeneic cell lines in practicing present invention provides the therapeutic advantage that, through administration of a cytokine-expressing allogeneic cell line and at least one additional cancer therapeutic agent (expressed by the same or a different cell line) to a patient with cancer, together with an autologous cancer antigen, paracrine production of an immunomodulatory cytokine, results in an effective immune response to a tumor. This obviates the need to culture and transduce autologous tumor cells for each patient, eliminating the problem of variable and inefficient transduction efficiencies.

### Bystander

In one further aspect, the present invention provides a universal immunomodulatory cytokine-expressing bystander cell line and a bystander cell line that expresses at least one additional cancer therapeutic agent. The same universal bystander cell line may express both a cytokine and cancer therapeutic agent or each may be expressed by a different universal bystander cell line. The universal bystander cell line comprises cells which either naturally lack major histocompatibility class I (MHC-I) antigens and major histocompatibility class II (MHC-II) antigens or have been modified so that they lack MHC-I antigens and MHC-II antigens. In one aspect of the invention, a universal bystander cell line is modified by introduction of a vector comprising a nucleic acid sequence encoding a cytokine operably linked to a promoter and expression control sequences necessary for expression thereof. In another aspect, the same universal bystander cell line or a second a universal bystander cell line is modified by introduction of a vector comprising a nucleic acid sequence encoding at least one additional cancer therapeutic agent operably linked to a promoter and expression control sequences necessary for expression thereof. The nucleic acid sequence encoding the cytokine and additional cancer therapeutic agent(s) may be introduced into the same or a different universal bystander cell line using the same or a different vector. The nucleic acid sequence encoding the cytokine or cancer therapeutic agent may or may not further comprise a selectable marker sequence operably linked to a promoter. Any combination of cytokine(s) and cancer therapeutic agent(s) that stimulate an anti-tumor immune response finds utility in the practice of the present invention. The universal bystander cell line preferably grows in defined, i.e., serum-free, medium, preferably as a suspension.

An example of a preferred universal bystander cell line is K562 (ATCC CCL-243; Lozzio et al., Blood 45(3): 321-334 (1975); Klein et al., Int. J. Cancer 18: 421-431 (1976)). A detailed description of the generation of human bystander cell lines is described for example in U.S. Pat. No. 6,464,973, expressly incorporated by reference herein.

Desirably, the universal bystander cell line expresses high levels of the cytokine, e.g., GM-CSF. Preferably, the universal bystander cell line expresses at least about 500 ng GM-CSF/10cells/24 hours.

In practicing the invention, the one or more universal bystander cell lines are incubated with an autologous cancer antigen, e.g., an autologous tumor cell (which together comprise a universal bystander cell line composition), then the universal bystander cell line composition is administered to the patient. Any suitable route of administration can be used to introduce a universal bystander cell line composition into the patient. Preferably, the composition is administered subcutaneously or intratumorally.

Typically, the cancer antigen is provided by (on) a cell of the cancer to be treated, i.e., an autologous cancer cell. In such cases, the composition is rendered proliferation-incompetent by irradiation, wherein the bystander cells and cancer cells are plated in a tissue culture plate and irradiated at room temperature using a Cs source, as detailed above.

The ratio of bystander cells to autologous cancer cells in a given administration will vary dependent upon the combination. With respect to GM-CSF-producing bystander cells, the ratio of bystander cells to autologous cancer cells in a given administration should be such that at least 36 ng GM-CSF/10 cells/24 hrs is produced. In general, the therapeutic effect is decreased if the concentration of GM-CSF is less than this. In addition to the GM-CSF threshold, the ratio of bystander cells to autologous cancer cells should not be greater than 1:1. Appropriate ratios of bystander cells to tumor cells or tumor antigens can be determined using routine methods in the art.

The use of bystander cell lines in practicing present invention provides the therapeutic advantage that, through administration of a cytokine-expressing bystander cell line and at least one additional cancer therapeutic agent (expressed by the same or a different cell line) to a patient with cancer, together with an autologous cancer antigen, paracrine production of an immunomodulatory cytokine, results in an effective immune response to a tumor. This obviates the need to culture and transduce autologous tumor cells for each patient, eliminating the problem of variable and inefficient transduction efficiencies.

### Evaluation of Combinations in Animal Models

B16F10 Melanoma Model

In one approach, the efficacy of cytokine-expressing cellular vaccine combination is evaluated by carrying out animal studies in the syngeneic B16F10 melanoma tumor model in the treatment setting. See, e.g., Griswold D P Jr., Cancer Chemother Rep 2;3(1):315-24, 1972 and Berkelhammer J et al., Cancer Res 42(8):3157-63, 1982. The murine melanoma cell line B16 is a well-defined cell line which is weakly immunogenic in syngeneic C57BL6 mice and therefore readily forms tumors in C57BL6 mice. Furthermore, several tumor associated antigens have been identified in this model which allow one to monitor tumor as well as antigen specific immune responses. Such well-characterized tumor cell lines are not readily available in other species. In addition, several murine-specific reagents are commercially available and are used to monitor anti-tumor immune responses in the various vaccine strategies. A typical study in the B16F10 melanoma tumor model makes use of at least 6 and generally 10-15 mice per group in order to obtain statistically significant results. Statistical significance is evaluated using the Student's t-test.

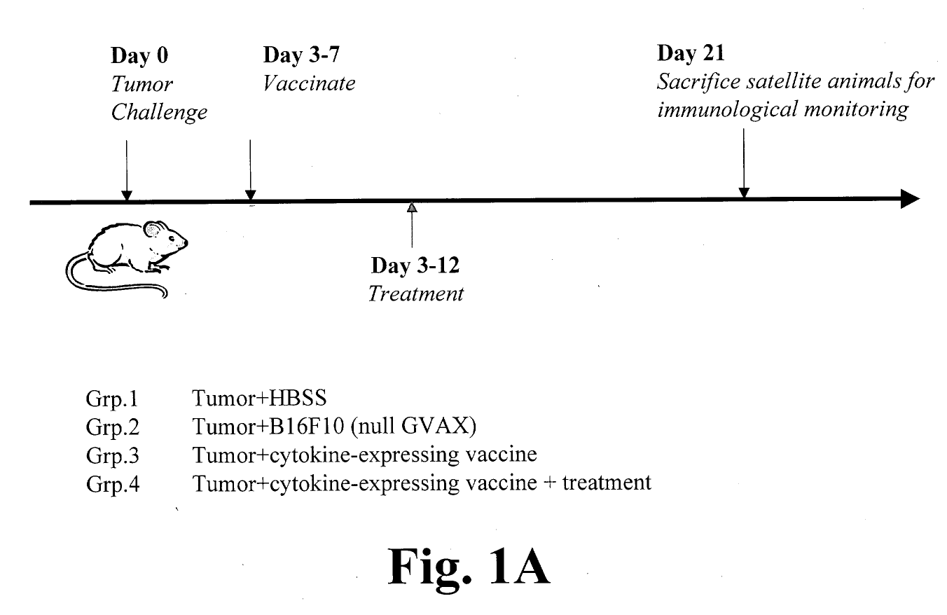

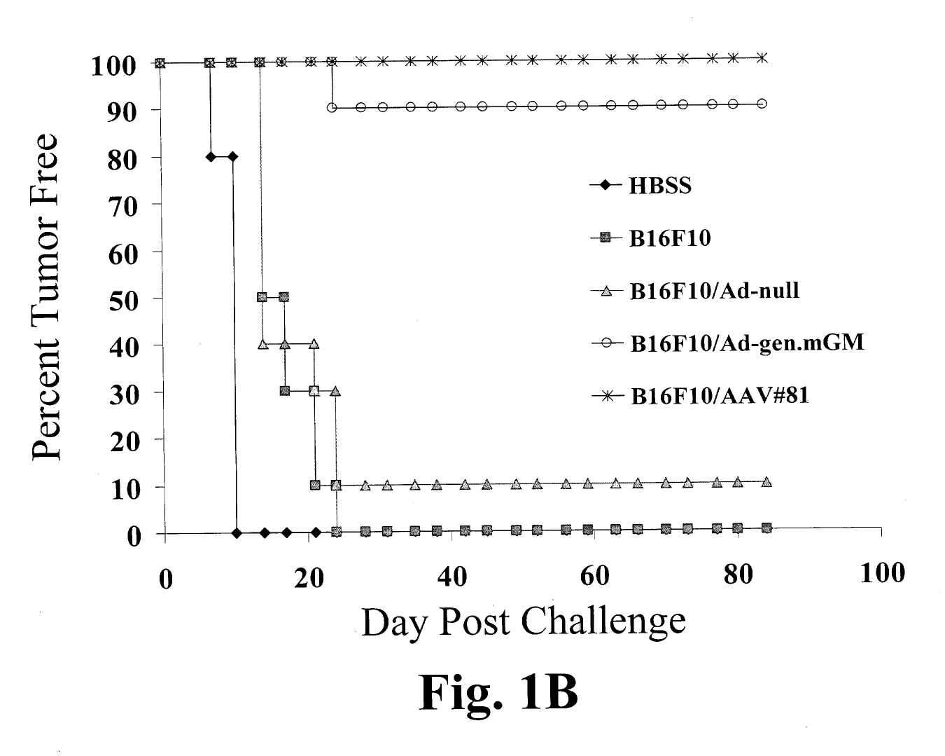

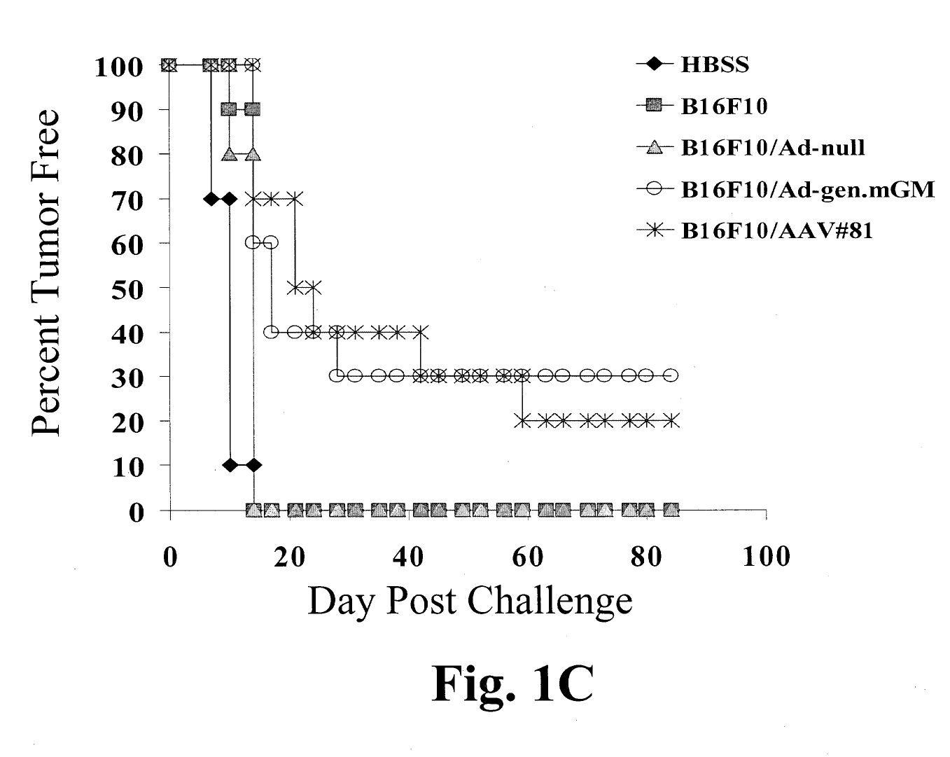

In carrying out studies using the B16F10 melanoma tumor model, female C57BL/6 mice are obtained from Taconic and mice are at 6-8 weeks old at the start of each experiment. In a typical experiment, mice are injected with 5×10⁴ B16B1/6 cells on day 0 via tail vein with a 27-gauge needle (FIG. 1A). After 14-21 days, mice are sacrificed and their tumor burden assessed by harvesting the mice lungs and counting the surface tumor metastasis and measuring the weight of the lung. An alternative B16F10 melanoma tumor model involves subcutaneous injection of B16F10 tumor cells. In this model, female C57B1/6 mice were injected subcutaneously in a dorsal/anterior location with 1×10⁵ B16F10 cells on day 0. On day 3, mice are vaccinated in a ventral/posterior location with 1-3×10⁶ irradiated (5000 rads) B16F1 or cytokine-expressing cellular vaccine (GVAX). Mice are followed for tumor development and survival.

A typical in vivo study in the B16F10 melanoma tumor model employs the following groups: HBSS only (negative control); irradiated B16F10/HBSS (control): cytokine-expressing cellular vaccine (GVAX)/HBSS; (cellular vaccine monotherapy control); additional cancer therapeutic agent or treatment (therapeutic agent/treatment monotherapy control); additional cancer therapeutic agent or treatment together with tumor antigen source; cytokine-expressing cellular vaccine plus cancer therapeutic agent or treatment.

Experiments in the syngeneic B16 melanoma model in C57BL6 mice, have shown that immunity was induced with B16 cells that were genetically modified to express GM-C SF, while non-transduced B16 cell were completely ineffective. Vaccination of C57BL6 mice with irradiated B16F10 melanoma cells engineered to secrete GM-CSF stimulates potent, long-lasting and specific anti-tumor immunity that prevents tumor formation in a majority of mice challenged with non-transduced B16F10 (prevention model, FIG. 1B). However, when irradiated GM-CSF-producing tumor cells are administered to mice harboring recently established subcutaneous tumors (treatment model; FIG. 1C), the protective anti-tumor immunity is less effective. Results from animal model experiments have convincingly shown that GM-CSF producing tumor cells are able to induce an immune response against the parental, non-transduced tumor cells.

Previous experiments have demonstrated that HBSS or irradiated B16F10 alone do not protect challenged mice from tumor formation. GM-CSF-expressing cellular vaccines (GVAX) alone were shown to protect from 30-50% of the challenged mice. The combination of GVAX plus at least one additional cancer therapeutic agent or treatment is expected to increase the efficacy of anti-tumor protection. The degree of protection depends on several factors such as the expression level of the additional cancer therapeutic agent and the cytokine-expressing cellular vaccine, the level of treatment (i.e. dose of the agent or the frequency and strength of radiation) and the relative timing and route of administration of the additional cancer therapeutic agent (e.g., as transfected cells or as a protein or chemical entity) relative to the timing of administration of the cytokine-expressing cellular vaccine, e.g., GVAX.

Furthermore, several tumor associated antigens have been identified which allow one to monitor tumor as well as antigen specific immune responses. For example, tumor antigen-specific T cells can be identified by the release of IFN-gamma following antigenic restimulation in vitro (Hu, H-M. et al., Cancer Research, 2002, 62; 3914-3919). Yet another example of new methods used to identify tumor antigen-specific T cells is the development of soluble MHC I molecules also known as MHC tetramers (Beckman Coulter (Immunomics). MHC tetramers are loaded with specific peptides that have been shown to be involved in an anti-tumor immune response. Examples within the B16F10 melanoma tumor model include but are not limited to gp100, Trp2, Trp-1, and tyrosinase. Similar melanoma-associated antigens have been identified in humans. Such tools provide information that can then be translated into the clinical arena.

RIP-Tag Spontaneous Pancreatic Islet Carcinoma Model

The RIP-Tag spontaneous pancreatic islet carcinoma model makes use of transgenic mice which have been genetically modified to express a rat insulin promoter (RIP) driven simian virus 40 (SV-40) antigen and develop islet cell carcinomas as a result of SV-40 oncogene expression in pancreatic islet cells. In the model, tumor development proceeds through a series ofwell-defined stages that occur over a period of 13.5 weeks in these mice. The RIP-Tag mice develop pancreatic insulinomas and islet cell carcinomas in a multi-step pathway that includes an angiogenic switch before solid tumor formation. 100% of the normal islets express the Tag oncogene however, do not show symptoms of dysplasia until 3-4 weeks of age. Hyperplastic cells, 50% of the islets, begin to appear by 10 weeks of age. Solid tumors emerge after 12-13 weeks that progress into large adenomas that sometimes develop into invasive carcinomas, characterized by high vasculature and dilated hemorrhagic vessels. (Bergers G et al., Science, 1999 Apr. 30;284(5415):808-12). The RIP-Tag spontaneous pancreatic islet carcinoma model is used to evaluate the efficacy of cytokine-expressing cellular vaccine combinations.

Assays for Efficacy of Combinations In Vivo Models

Tumor burden is assessed every three days after tumor challenge. Typically, on day 18, spleens cells are assessed for CTL activity by in vitro whole cell stimulation for 5 days. Target cells are labeled with ⁵¹Cr and co-incubated with splenic effector CTL and release of ⁵¹Cr into the supernatants as an indicator of CTL lysis of target cells. On day 3 of in vitro stimulated CTL supernatants are tested for IFN-gamma production by CTL. In brief, wells are coated with coating antibody specific for IFN-gamma, supernatant is then added to wells, and IFN-gamma is detected using an IFN-gamma specific detecting antibody. IFN-gamma can also be detected by flow cytometry, in order to measure cell-specific IFN-gamma production.

Xenogen Imaging of Tumor Models

In some studies, in vivo luminescence of tumor bearing mice is monitored by biweekly monitoring of B16-F10-luciferase (Xenogen Inc.) injected mice. In brief, Balb/c nu/nu mice are injected with 5×10⁴ or 2×10⁵ cells of B16F10-luc cells via tail vein on day 0. Mice are monitored for tumor burden when necessary by intra-peritoneal injection of excess luciferin substrate at 1.5 mg/g mice weight. Twenty minutes after substrate injection, mice are anesthesized and monitored for in vivo luminescence with Xenogen IVIS Imaging System (Xenogen Inc.) luminescence sensitive CCD camera by dorsal or ventral position. Data is collected and analyzed by Living Image 2.11 software.

GVAX Combinations

The present invention is directed to combinations of a cytokine-expressing cellular vaccine (e.g., GVAX) plus at least one additional cancer therapeutic agent or treatment. The agent or treatment may be a chemotherapeutic agent, an agent that modulates the immune response to a cancer antigen, radiation, etc. Exemplary embodiments of the invention, include, but are not limited to GVAX plus one or more of the following: an anti-CTLA-4 monoclonal antibody; an anti-41BB mAb; anti-CD40 or membrane/soluble CD40L; interferon alpha (IFN-alpha); IL-2 or IL-18; a HSP (heat shock protein); CpG (dinucleotides that mimic bacterial DNA); anti-OX-40 or soluble/membrane bound OX-40 Ligand; Cox-2 inhibitors; Iressa; anti-epidermal growth factor R (EGF-R); anti-VEGF R or TRAIL. Exemplary combinations are described in more detail below.

Co-Stimulatory Molecules in Combination with GVAX

In natural immune responses, CD4+ T helper (Th) cells, reactive with peptide antigens presented by MHC class II molecules on dendritic cells (DC), can drive the maturation of DC which is required for induction of CD8+ CTL immunity. Proper induction, expansion and maintenance of CTL responses are achieved through the interaction between CD4+ T cells, DC and CD8+ T cells. The cells to a large extent operate through up-regulation of CD40L, which interacts with DC-expressed CD40 to effect DC maturation. CD80/CD86 expressed by mature or activated DC can effect CTL induction by interaction with the CD28 costimulatory receptor on CD8+T cells. For maintenance and full expansion of CTL, interaction of the DC-expressed 4-1BB ligand with its receptor 4-1BB on CTL is also important. DC activation may be triggered by e.g., agonistic anti-CD40 antibody or ligands of Toll-like receptors (TLR) such as LPS (TLR4 ligand) or oligodeoxy-nucleotides containing CpG-motifs (TLR9 ligand).

(1) Cytokine-Expressing Cellular Vaccines (e.g., GVAX) plus Anti-CTLA-4 Monoclonal Antibody

The CTLA4 molecule, expressed at low levels on resting T cells and induced by T cell activation, transduces a negative signal to T cells which blocks IL-2 production, IL-2R expression, and induces cell cycle arrest. CTLA4 regulates both CD28 and non-CD28 mediated T cell activation, and has an important role in normal immune homeostasis (Bour-Jordan et al., Nature Immunol. 4: 182-188, 2003). In animal models, anti-CTLA-4 antibody can induce tumor rejection in immunogenic tumors, and in combination with anti-tumor vaccination, can induce rejection of minimally immunogenic tumors. Clinical trials have been initiated with a fully human antibody that binds to human CTLA-4 for treatment of metastatic melanoma and hormone refractory prostate carcinoma (HRPC) patients.

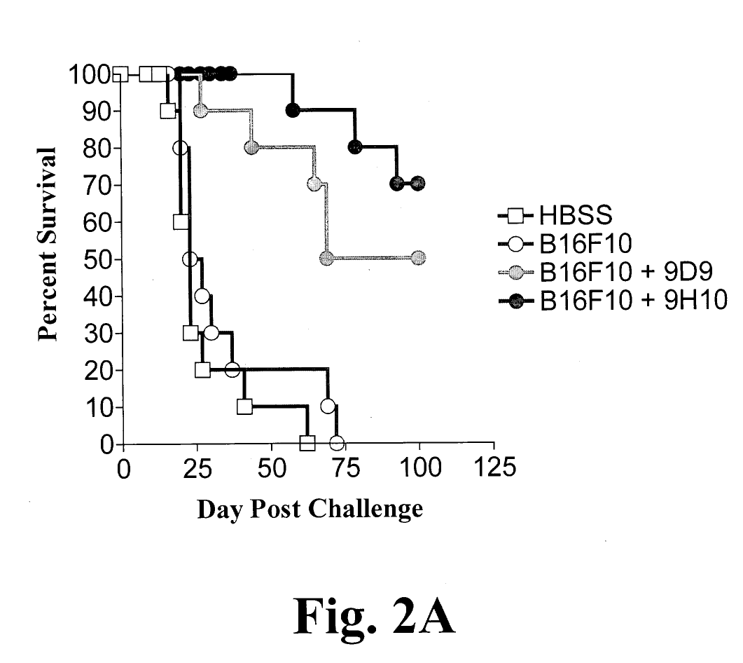

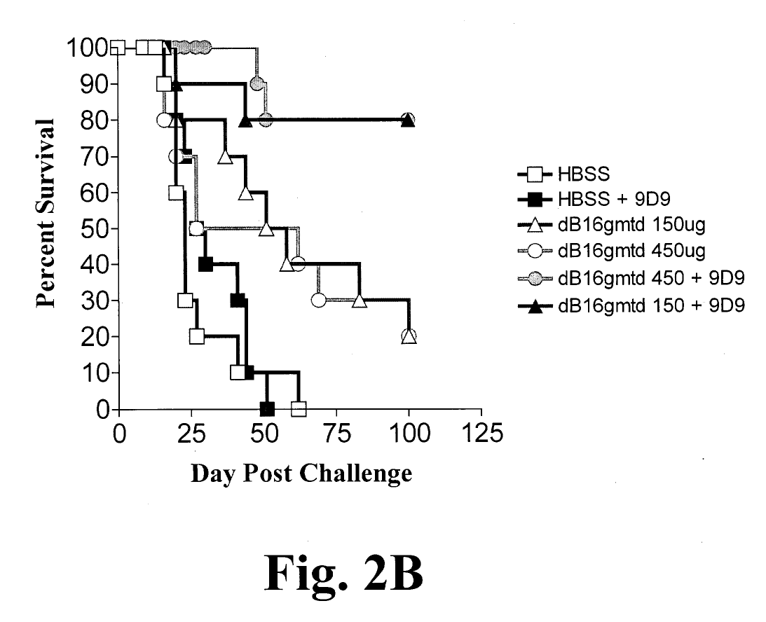

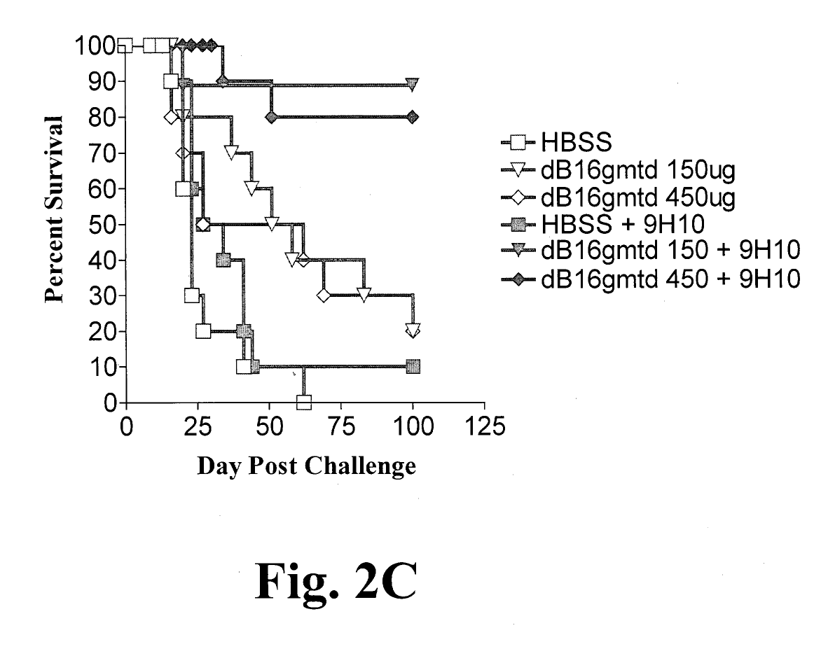

Previous reports indicate that anti-CTLA-4 mAb or GM-CSF-secreting cellular vaccines provide partial protection to mice when either is used as a monotherapy for non-immunogenic tumors such as B16 melanoma. The results presented here in demonstrate that the combination of GM-CSF-secreting B16 tumor cells and anti-CTLA-4 acts synergistically, resulting in highly protective anti-tumor immune responses. In order to achieve the maximal synergistic effect of these two biologics in clinical trials, careful evaluation of treatment regimens must be carried out in preclinical studies. Example 1 details studies where a murine anti-mouse anti-CTLA-4 mAb (9D9), which blocks B7/CTLA-4 interactions was tested in the B16 melanoma tumor model with and without vaccination with a cytokine-expressing cellular vaccine (GM-CSF-secreting B16F10 tumor cells; B16-GM) (FIG. 2A). The anti-CTLA-4 mAb was ineffective as a monotherapy in protecting C57B1/6 mice from a B16F10 tumor challenge. In contrast, 80% of the mice treated with B16-GM plus the anti-CTLA4 antibody, 9D9, were protected, demonstrating that 9D9 is biologically active and further supports the concept that anti-CTLA-4 and GVAX vaccines are an effective anti-cancer combination therapy (FIG. 2B).

These results demonstrate that in practicing the present invention an autologous, allogeneic, or bystander cytokine-expressing cellular vaccine (e.g., GVAX) may be administered to a cancer patient in combination with an anti-CTLA-antibody resulting in enhanced therapeutic efficacy relative to either monotherapy alone.

(2) Cytokine-Expressing Cellular Vaccines (e.g., GVAX) plus anti-41BB mAb CD137, also known as 4-1BB, is a member of the TNFR superfamily (Kwon BS et al., Cell Immunol. 121(2):414-22, 1989; Kwon BS et al., Proc Natl Acad Sci U S A. 86(6):1963-7, 1989). CD137 is expressed as monomers, dimers, and tetramers on the surface of activated T cells around day 4-6 after activation through the TCR complex. Interaction of CD137 with its ligand, 4-1BBL, is reported to deliver a costimulatory signal leading to expansion, cytokine production, including IFN-g, development of CTL and inhibition of activation induced cell death (AICD). 4-1BB is expressed on activated T cells with the peak of expression 2-3 days post activation. The ligand for 4-1BB (4-1BBL) is expressed on activated APC. Several studies have demonstrated that administration of anti-4-1BB monoclonal antibody can increase CTL responses and/or eradicate established tumors in vivo. See, e.g., Shuford et al. J. Exp. Med.; 1997 and Melero et al., Nature medicine; 1997.

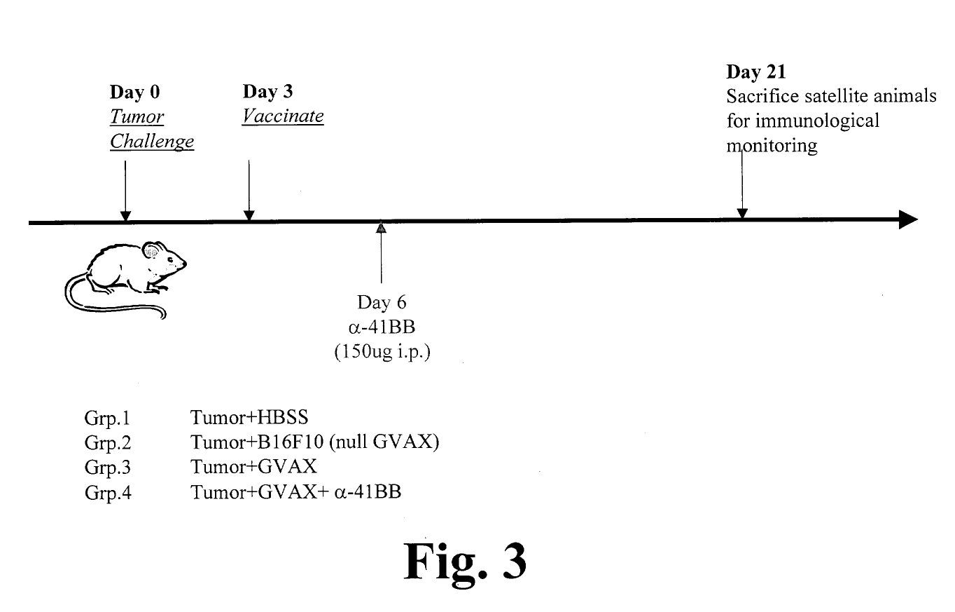

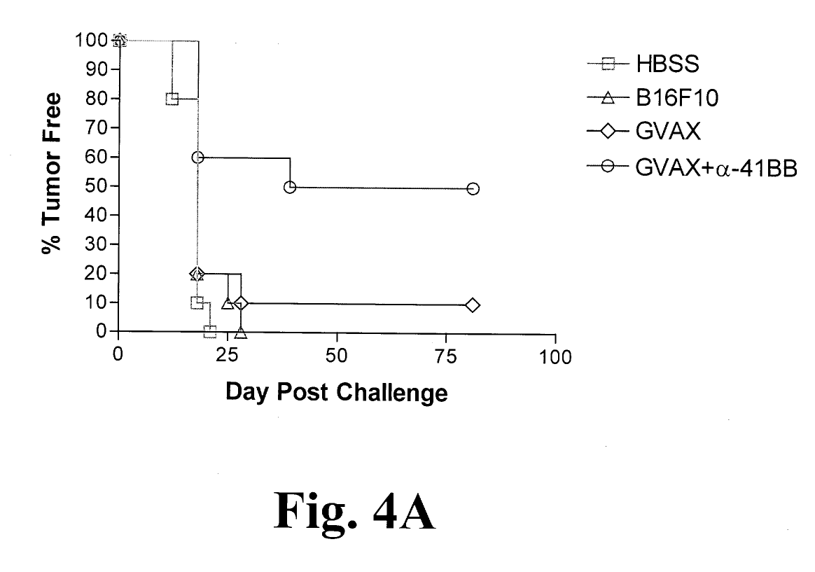

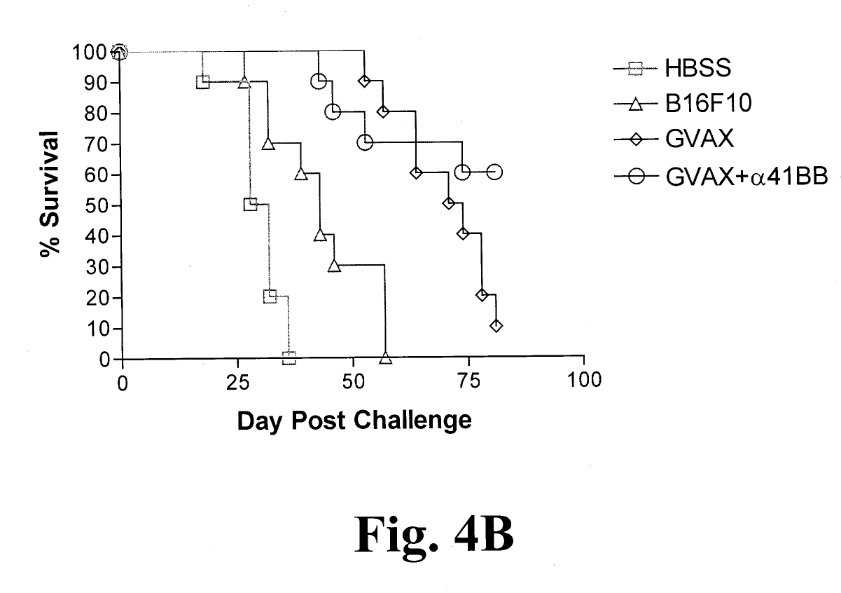

4-1BB ligand is described in U.S. Pat. No. 5,674,704; 4-1BB ligand polypeptides and a cell surface receptor that binds 4-1BB ligand are described in U.S. Pat. No. 6,355,779; monoclonal antibodies (mAb) which specifically bind to the extracellular domain of 4-1BB are described in U.S. Pat. Nos. 6,210,669 and 5,928,893; a humanized antibody specific for human 4-1BB is described in U.S. Pat. No. 6,458,934; and a method to enhance T cell proliferation by administering an agonistic mAb which binds to 4-1BB is described in U.S. Pat. No. 6,303,121, each of which is expressly incorporated by reference herein. As detailed in Example 2, the efficacy of cytokine-expressing cellular vaccine/4-1BB combinations was evaluated in the B16F10 tumor model (FIG. 3) and the results show that the combination enhanced both the number of tumor free mice (FIG. 4A) and survival (FIG. 4B) and for up to 80 days post-challenge.

These results demonstrate that in practicing the present invention an autologous, allogeneic, or bystander cytokine-expressing cellular vaccine (e.g., GVAX) is administered to a cancer patient in combination with an anti 4-1BB antibody resulting in enhanced therapeutic efficacy relative to either monotherapy alone.

(3) Cytokine-Expressing Cellular Vaccines (e.g., GVAX) plus anti-CD40 or Membrane/Soluble CD40L

CD40L binds to CD40. CD40 is expressed on B cells and dendritic cells. CD40 has a central role in B-cell growth, B cell differentiation and B cell survival. Signaling thru CD40 rescues B cells from apoptosis induced by FAS. It also induces B cells to undergo Ig isotype switching and to express CD80 (B7 or B7.1).

Antigen presenting cells (APCs), including specialized dendritic cells (DC), play a pivotal role in the direct activation and differentiation of T cells by providing co-stimulatory signals and cytokines involved in the development of cellular immune responses. Granulocyte-macrophage colony stimulatory factor (GM-CSF), a cytokine produced by fibroblasts, endothelial cells, T cells, and macrophages, activates the processing and antigen-presenting function of DC. Irradiated B16F10 melanoma cells transduced to express GM-CSF (B16-GM) confer partial protective anti-tumor immunity to recently established subcutaneous B16F10 tumors. Membrane-bound CD40 ligand (CD40L) and anti-CD40 monoclonal antibody have been shown to activate DCs. CD40, a member of the tumor necrosis factor receptor (TNF-R) family, is a surface receptor expressed on antigen presenting cells (APCs) including, B cells, DCs, and activated monocytes.

The natural ligand for CD40 is CD40L. CD40L enhances the antigen presentation function of CD40-expressing B cells and DCs, resulting in the production of IL-12, which plays a critical role in the development of effector T cells.

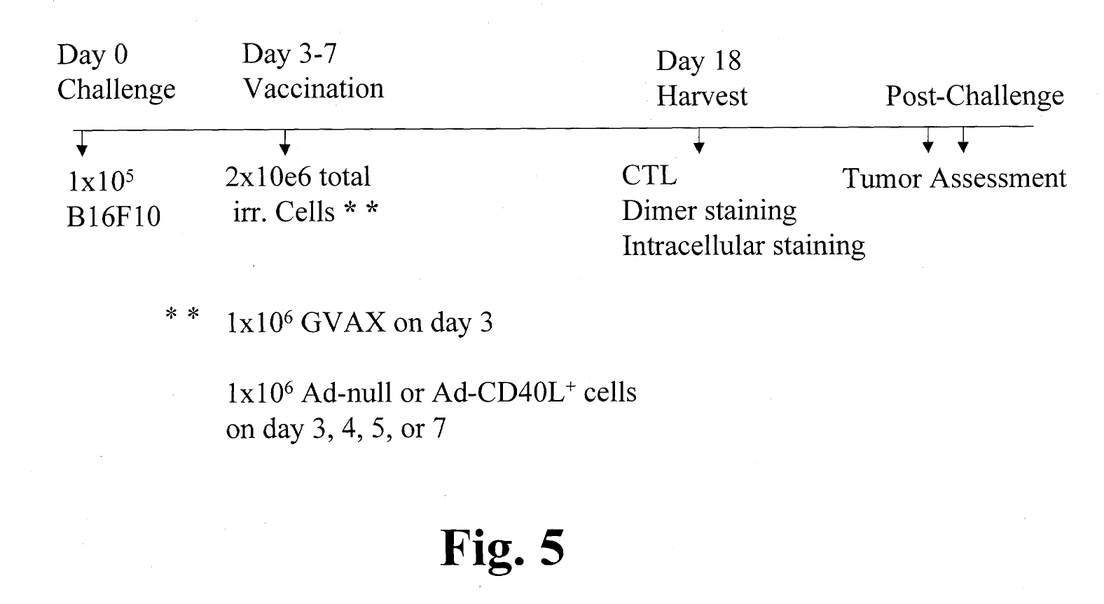

Example 3 details in vivo studies directed to evaluate cytokine-expressing cellular vaccines in combination with either anti-CD40 or membrane bound CD40 ligand (CD40L) (See FIG. 5). Experimental results show that B16F10 cells transduced by adenovirus to express CD40L, given in combination with B16-GM, significantly increased the survival of mice challenged with B16F10 melanoma, when administered 4 days after vaccination with B16-GM. CD40L administered the same day with B16-GM was shown to exacerbate the disease. To further demonstrate the efficacy of B16-GM and anti-CD40 combinations, animals were treated with an anti-CD40 monoclonal antibody administered at different times post vaccination. Similar to results obtained for CD40L, the timing of anti-CD40 administration with respect to vaccination was critical to efficacy. The data indicate that targeting DCs can enhance the potency of vaccination with GM-CSF secreting cellular vaccines.

The efficacy of a cytokine-expressing cellular vaccine/CD40L and anti-CD40 combinations were evaluated in the B16F10 tumor model (as further described in Example 3). In practicing the present invention an autologous, allogeneic, or bystander cytokine-expressing cellular vaccine (e.g., GVAX) is administered to a cancer patient in combination with an anti-CD40 antibody or CD40 ligand expressed by autologous, allogeneic, or bystander cells, resulting in enhanced therapeutic efficacy relative to either monotherapy alone.

(4). Cytokine-Expressing Cellular Vaccines (e.g., GVAX) Plus One Or More Additional Cytokines

In one aspect of the invention, a cytokine-expressing cellular vaccine (e.g., GVAX) is administered to a patient in combination with at least one additional cytokine for the treatment of cancer. Exemplary cytokines include, but are not limited to IL-2 through 11-29, r-IFN, TNF-alpha, CD2, MIP3a and ICAM.

Cytokine-Expressing Cellular Vaccines (e.g., GVAX) Plus Interferon-Alpha

Interferons were identified in 1957 as a factor that interferes with viral infection. Type I (IFN-a and IFN-b) are produced during the early phase of viral infection. Type II (IFN-g) is important in the development of cellular immune responses. IFN-a has been shown to have a direct anti-proliferative effect on tumor cells via cytotoxic effects (apoptosis). IFN-a is known to modulate antigen specific immunity by changing the microenvironment, which favors the proliferation of Th1 lymphocytes. IFN-a is important for the generation of CTLs, by promoting clonal expansion and survival of CD8+ T cells.

IFN-a is approved for the clinical treatment of lymphoma, leukemia (hairy cell), Kaposi's sarcoma, and melanoma. IFN-a exhibits potent immunomodulatory properties that promote Th1 lymphocyte development, generation of tumor-antigen specific CTL, CD8+ T cell activation/survival and dendritic cell activation. Furthermore, IFN-a directly affects tumor growth by inhibiting proliferation and inducing apoptosis and indirectly by inhibiting tumor angiogenesis.

Studies were undertaken to investigate the effect of the combination of a cytokine-expressing cellular vaccine (GM-CSF-secreting B16F10 tumor cells; B16-GM) together with IFN-a (detailed in Example 4). Recombinant murine IFN-a (rmIFN-alpha) given as a monotherapy protected up to 30% of mice challenged with B16F10 tumor cells when it was administered subcutaneously at the tumor challenge site, but not when administered at a distant site. Similarly, 30-40% of animals that received B16-GM alone remained tumor free after challenge. Interestingly, administration of the combination of IFN-alpha and B16.GM resulted in 70-80% tumor free survival. These effects were observed following multiple IFN-alpha dosing regimens (20000 U total dose) including four daily injections of 5000 U or a metronomic regimen that consisted of 500 U/day, 5 days/week for 8 weeks. Furthermore, re-challenge of surviving animals with a lethal dose of B16F10 100 days after the primary challenge demonstrated that only B16-GM-vaccinated mice (with or without IFN-alpha) survived the re-challenge. These data suggest that the synergistic effect resulting in increased survival after combination therapy is not due to increased tumor-specific memory responses. In summary, the results demonstrate that IFN-alpha combined with a GM-CSF secreting cellular vaccine results in increased survival in the B16F10 model, and may represent a new approach to combination tumor immunotherapy.

In one preferred embodiment of the invention, the additional cancer therapeutic agent is interferon-alpha, which is administered together with a GM-CSF-expressing cellular vaccine. In practicing the present invention, IFN-alpha or an interferon-alpha expressing cell line is administered in combination with an autologous, allogeneic, or bystander cytokine-expressing cellular vaccine (e.g., GVAX).

Interferon-alpha may be administered in the form of the protein or in the form of an autologous, allogeneic, or bystander interferon-alpha-expressing cell line. Exemplary interferon-alpha sequences for use in practicing the invention may be found for example at GenBank Accession Nos. NM1FNA1, NM1FNA2, expressly incorporated by reference herein.

Cytokine-Expressing Cellular Vaccines (e.g., GVAX) Plus IL-2

Previous studies have shown that transfection of a gene encoding IL-2 into a tumor cell stimulated an MHC class I-restricted cytolytic T lymphocyte (CTL) response against the tumor in vivo, suggesting that IL-2 can play a role in enhancing the immune responsiveness to a tumor in vivo (Frost et al., WO 92/05262; Fearon et al., Cell 1990 Feb. 9;60(3):397-403).

In a preferred embodiment, the invention provides a cytokine-expressing cellular vaccine combination wherein the combination comprises IL-2, administered together with a GM-CSF-expressing cellular vaccine. IL-2 may be administered in the form of the protein or in the form of an autologous, allogeneic, or bystander IL-2-expressing cell line.

An exemplary IL-2 sequence for use in practicing the invention may be found for example at GenBank Accession No. NMIL04, NM_{—}000586, expressly incorporated by reference herein.

Previous studies in the B16 melanoma model have shown that cells expressing both IL-2 and GM-CSF can generate systemic immunity and enhanced survival, as described in WO 00/72686, expressly incorporated by reference herein.

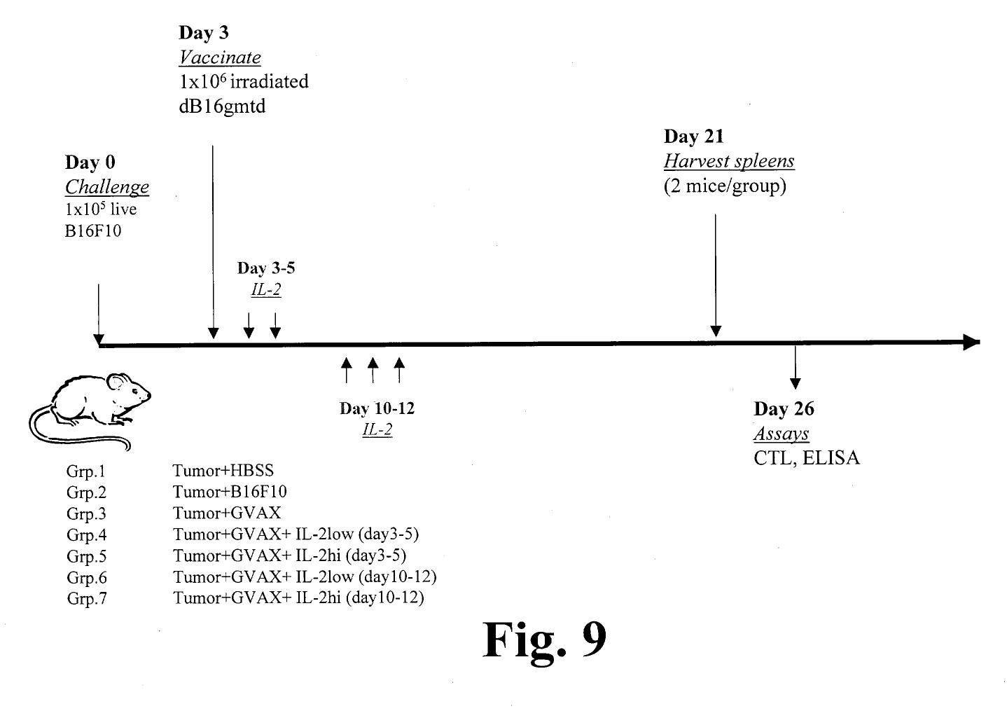

The efficacy of a cytokine-expressing cellular vaccine/IL-2 combinations has been evaluated in the B16F10 tumor model (as shown in FIG. 9; WO 00/72686). In practicing the present invention an autologous, allogeneic, or bystander cytokine-expressing cellular vaccine (e.g., GVAX) is administered to a cancer patient in combination with IL-2 protein or as an autologous, allogeneic, or bystander IL-2-expressing cell line.

(6) Cytokine-Expressing Cellular Vaccines (e.g., GVAX) plus HSP (Heat Shock Protein)

Recent evidence suggests that heat shock proteins (HSPs) associate with a broad array of self-peptides that can be presented on MHC I molecules through a mechanism called cross-presentation. Additionally, HSPs may activate professional APC through specific receptor interactions that stimulate pro inflammatory cytokines and upregulation of costimulatory molecules. As a result tumor derived HSPs are potent stimulators of tumor-specific immune responses.

The efficacy of a cytokine-expressing cellular vaccine/HSP combinations are evaluated in the B16F10 tumor model (as further described in Example 7). A HSP may be administered in the form of the protein or in the form of an autologous, allogeneic, or bystander HSP-expressing cell line. An exemplary HSP sequence for use in practicing the invention may be found for example at GenBank Accession No. AX194370, expressly incorporated by reference herein.

In practicing the present invention an autologous, allogeneic, or bystander cytokine-expressing cellular vaccine (e.g., GVAX) is administered to a cancer patient in combination with HSP or as an autologous, allogeneic, or bystander HSP-expressing cell line.

(7) Cytokine-Expressing Cellular Vaccines (e.g., GVAX) plus CpG

Mammalian DNA contains CG-at a low frequency and even when present, the 5-position of cytosine is methylated so that it is not recognized by the mammalian immune system, In contrast, bacterial DNA contains a large number of unmethylated CG-dinucleotides which are recognized by the vertebrate immune system. CpG ODN have been shown to exhibit potent immunostimulatory properties that enhance the function of both the innate and adaptive immune response. CpG oligos provide enhanced stimulation of Th1 cyotkine production, activation of antigen presenting cells such as dendritic cells and macrophages, and induce increased IFN-a production, thereby promoting a variety of anti-cancer immune effects. The combination of CpG adjuvant with a 35 amino acid long synthetic peptide comprising both tumor-specific CTL and Th epitopes proved to be a highly effective vaccine formulation capable of inducing therapeutic immunity against human papillomavirus-induced mouse tumors (Kim et al. Cancer Res. 62(24):7234-40. 2002).

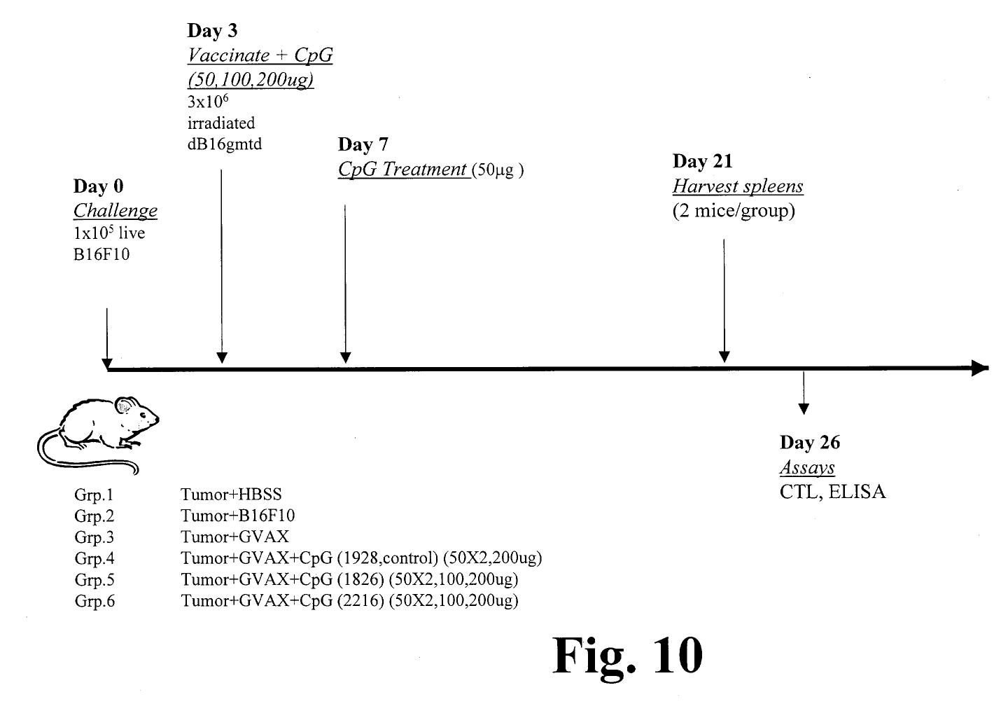

The combination of a cytokine-expressing cellular vaccine (i.e., GVAX) and CpGs may lead to greater overall protection than the individual therapies. The efficacy of a cytokine-expressing cellular vaccine/CpG combination is evaluated in the B16F10 tumor model (as shown in FIG. 10). In practicing the present invention an autologous, allogeneic, or bystander cytokine-expressing cellular vaccine (e.g., GVAX) is administered to a cancer patient in combination with a CpG dinucleotide.

(8) Cytokine-Expressing Cellular Vaccines (e.g., GVAX) plus anti-OX40 mAb or Membrane/Soluble OX40L.

OX40 (CD134), a member of the TNFR superfamily, is expressed on activated CD4⁺ T cells. Crosslinking OX40 on the surface of CD4 T cells generates a potent costimulatory signal that enhances T-cell proliferation to submitogenic concentrations of Con A, anti-CD3, PHA and PMA. Antigen-specific T-cell responses can also be enhanced by OX40-mediated costimulation. OX40 engagement in vivo inhibits the peripheral deletion of CD4+ T cells that occurs after their expansion in response to superantigens or soluble protein Ag, and OX40 knockout mice are deficient in generating normal levels of Ag-specific memory T cells following immunization in vivo. Thus, OX40 engagement appears to be important for the generation and maintenance of memory T cells following in vivo immunization. The combination of GM-CSF-producing tumor cells and stimulation through the OX40 receptor (OX40R) should provide an enhanced therapeutic effect towards cancer than the individual therapies. The efficacy of a cytokine-expressing cellular vaccine/OX40L or anti-OX40 combinations is evaluated in syngeneic tumor models (as further described in Example 5 and Gri, G. et al. J. Immunol. 2003, Jan 1;170(1):99-106). In practicing the present invention an autologous, allogeneic, or bystander cytokine-expressing cellular vaccine (e.g., GVAX) is administered to a cancer patient in combination with either anti-OX40 antibody or as an autologous, allogeneic, or bystander OX40L-expressing cell line.

(9) Cytokine-Expressing Cellular Vaccines (e.g. GVAX) plus Cox-2 inhibitors

Cyclooxygenase (COX) (also known as PGH synthase or prostaglandin endoperoxide synthase), is the key enzyme catalyzing the biosynthesis of Prostaglandins (PGs). There are two genes that express two distinct isoforms of the enzyme COX-1 and COX-2, which have similar primary protein structure (60% homology) and catalyze essentially the same reaction. Prostaglandins are involved in as diverse normal processes as renal function, vasomotor tone, platelet aggregation and blood clotting, differentiation of immune cells, wound healing, nerve growth, bone metabolism, ovulation, and initiation of labor. COX-2, is an inducible isoform that is found mainly in inflammatory and immune cells (neutrophils, macrophages, mast cells, etc). Pro-inflammatory cytokines and growth factors induce COX-2, which suggests that it may play an important role in the process of inflammation and in the control of cell growth. COX-2 is strongly expressed in human colon cancer cells, and is thought to delay the progress of colon tumors possibly by causing apoptosis (programmed cell death) of the tumor cells. See, e.g., Vane J R et al., Annu Rev Pharmacol Toxicol, 1998, 38:, 97-120 and Cryer B and Feldman M., Am J Med, 1998 May, 104:5, 413-21.

In practicing the present invention an autologous, allogeneic, or bystander cytokine-expressing cellular vaccine (GVAX) is used in combination with an autologous, allogeneic, or bystander COX-2-expressing cell line.

E. Cytokine-Expressing Cellular Vaccines (e.g., GVAX) plus Anti-Angiogenic Agents

It is generally accepted that tumor development requires the secretion by cancer cells of soluble mediators, so-called “tumor angiogenic factors”, which activate the formation of new blood vessels. The discovery of vascular endothelial growth factor (VEGF) and of new proteases had led to the identification of compounds involved in tumor angiogenesis and has allowed for the design of new therapeutic strategies, including the use of anti-angiogenic compounds.

In one aspect, the invention provides a cytokine-expressing cellular vaccine combination comprising an anti-angiogenic compound. Preferred anti-angiogenic compounds include endostatin, angiostatin, platelet factor-4, the 16-kD fragment of prolactin, sFlt-1 (a soluble fragment of fms-like tyrosine kinase 1 receptor), sKDR (soluble fragment of kinase insert domain receptor), sVEGFR3 (soluble extracellular forms of VEGFRs), anti-epidermal growth factor R (EGF-R), TRAIL (tumor necrosis factor—related apoptosis-inducing ligand), thrombospondin, interferon alpha and (PEDF) pigment epithelium-derived factor, as well as factors involved in signaling pathways that lead to expression/production of these compounds.

In practicing the present invention an autologous, allogeneic, or bystander cytokine-expressing cellular vaccine (GVAX) is administered to a cancer patient in combination an anti-angiogenic agent delivered in the form of a protein or as an autologous, allogeneic, or bystander anti-angiogenic agent-expressing cell line.

Delivery of Cytokine-Expressing Cellular Vaccines to the Patient

The present invention provides methods for cancer therapy, where a cytokine-expressing cellular vaccine and at least one additional cancer therapeutic agent or treatment are administered to a cancer patient. Desirably, the method effects a systemic immune response, i.e., a T-cell response and/or a B-cell response, to the cancer.

In a preferred aspect of the methods described herein, a cytokine-expressing cellular vaccine combination is administered to a cancer patient, wherein the cytokine-expressing cellular vaccine comprises mammalian, preferably human tumor cells, and the cells in the cytokine-expressing cellular vaccine are rendered proliferation incompetent, such as by irradiation. Administration of a cytokine-expressing cellular vaccine combination results in an enhanced immune response to the cancer as compared to the immune response to the same cancer following administration of the cytokine-expressing cellular vaccine or cancer therapeutic agent or treatment component of the combination alone. In other words, the combined administration of a cytokine-expressing cellular vaccine and at least one additional cancer therapeutic agent or treatment described above results in enhanced therapeutic efficacy as compared to administration of a cytokine-expressing cellular vaccine alone or administration of the cancer therapeutic agent(s) or treatment(s) alone.

The cytokine-expressing cellular vaccine combination may be administered by any suitable route. Preferably, the composition is administered subcutaneously or intratumorally. Local or systemic delivery can be accomplished by administration comprising administration of the combination into body cavities, by parenteral introduction, comprising intramuscular, intravenous, intraportal, intrahepatic, peritoneal, subcutaneous, or intradermal administration. In the event that the tumor is in the central nervous system, the composition must be administered intratumorally.

In one exemplary embodiment, the cytokine-expressing cellular vaccine is GVAX, where the cytokine expressed is GM-CSF and the at least one additional cancer therapeutic agent or treatment is an antibody to CTLA4.

In a further exemplary embodiment, the cytokine-expressing cellular vaccine is GVAX, where the cytokine expressed is GM-CSF and the at least one additional cancer therapeutic agent or treatment is an antibody to 4-1BB.

In a still further exemplary embodiment, the cytokine-expressing cellular vaccine is GVAX, where the cytokine expressed is GM-CSF and the at least one additional cancer therapeutic agent or treatment is an antibody to CD40 or membrane bound/soluble CD40 ligand.

In a still further exemplary embodiment, the cytokine-expressing cellular vaccine is GVAX, where the cytokine expressed is GM-CSF and the at least one additional cancer therapeutic agent or treatment is an antibody to OX40 or membrane bound/soluble OX40 ligand.

In yet another exemplary embodiment, the cytokine-expressing cellular vaccine is GVAX, where the cytokine expressed is GM-CSF and the at least one additional cancer therapeutic agent or treatment is interferon-alpha.

In another exemplary embodiment, the cytokine-expressing cellular vaccine is GVAX, where the cytokine expressed is GM-CSF and the at least one additional cancer therapeutic agent or treatment is IL-2.

As will be understood by those of skill in the art, the optimal treatment regimen will vary. As a result, it will be understood that the status of the cancer patient and the general health of the patient prior to, during, and following administration of a cytokine-expressing cellular vaccine combination, the patient will be evaluated in order to determine if the dose of each component and relative timing of administration should be optimized to enhance efficacy or additional cycles of administration are indicated. Such evaluation is typically carried out using tests employed by those of skill in the art to evaluate traditional cancer chemotherapy, as further described below in the section entitled “Monitoring Treatment”.

In one preferred aspect of the methods described herein, cytokine-expressing cellular vaccine is administered to the patient prior to, or following, but not at the same time as administration of the additional cancer therapeutic agent or treatment.

In another preferred embodiment a GM-CSF-expressing cellular vaccine is administered to the patient prior to administration of an anti-CD40 antibody or CD40L.

Delivery of Chemotherapeutic Agents

In some aspects of the invention, the cytokine-expressing cellular vaccine combination comprises a chemotherapeutic agent. An important consideration in this aspect of the invention is effective delivery of the cancer therapeutic agent in a pharmaceutically acceptable carrier.

In accordance with this aspect of the invention, the choice of cancer therapeutic (i.e. chemotherapeutic) agent or agents and the corresponding route and timing of delivery takes advantage of one of more of: (i) established use in treatment of the particular type of cancer under treatment; (ii) the ability of the selected agent to result in an improved therapeutic outcome when administered in combination with the cytokine-expressing cellular vaccine; and (iii) delivery of the agent by a mode of administration effective to achieve sufficient localized exposure of the agent to cancer cells.

Typically, the cancer therapeutic agent is administered by a route and using a treatment regimen that has an established use in cancer therapy. As set forth above, the optimal route will vary with the cancer therapeutic agent. Local or systemic delivery can be accomplished by administration into body cavities, inhalation or insufflation of an aerosol, or by parenteral introduction, comprising intramuscular, intravenous, intraportal, intrahepatic, peritoneal, subcutaneous, or intradermal administration. However, preferred routes typically include slow intravenous infusion (IV drip), oral administration and local injection. In the event that the tumor is in the central nervous system, the composition must be administered intratumorally. The formulations are easily administered in a variety of dosage forms such as injectable solutions, drug release capsules, implants or in combination with carriers such as liposomes or microcapsules.

Parenteral administration may be accomplished using a suitable buffered aqueous solution and the liquid diluent which has been prepared in isotonic form using saline or glucose. Such aqueous solutions are appropriate for intravenous, intramuscular, subcutaneous and intraperitoneal administration. (See, for example, “Remington's Pharmaceutical Sciences”, 15th Edition, pages 1035-1038 and 1570-1580). Sterile injectable solutions are prepared by incorporating the chemotherapeutic agent in the required amount of an appropriate solvent with various other ingredients included, followed by filter sterilization. Sterile powders for use in sterile injectable solutions may be prepared by vacuum drying or freeze drying techniques or other means to result in a powder of the active chemotherapeutic agent plus additional desired ingredients prepared from a previously sterile solution.

For example, when orally administered, the cancer therapeutic agent may be combined with an inert diluent or in an edible carrier, or enclosed in hard or soft shell gelatin capsules, compressed into tablets, incorporated directly into food, incorporated with excipients and used in the form of ingestible tablets, buccal tables, troches, capsules, elixirs, suspensions, syrups, wafers, and the like. The appropriate amount of cancer therapeutic agent is specific to the particular therapeutic agent and is generally known in the art.

Recommended dosages and dosage forms for a large number of cancer therapeutic agents have been established and can be obtained from conventional sources, such as the Physicians Desk Reference, published by Medical Economics Company, Inc., Oradell, N.J. Typically, the optimal route of delivery has been determined for known cancer therapeutic agents by well-established procedures and analysis, e.g., in clinical trials.

It will be understood that the invention contemplates treatment regimens that include the administration of one or more cancer therapeutic agents and administration of a cytokine-expressing cellular vaccine for therapy of cancer. Such a treatment regimen may be administered prior to, contemporaneously with, or subsequent to an additional cancer treatment, such as radiation therapy, further chemotherapy and/or immunotherapy.

The present invention provides the advantage that the dose of the one or more cancer therapeutic agents or treatments may be decreased when administered together with a cytokine-expressing cellular vaccine relative to treatment regimens that do not include cytokine-expressing cellular vaccine administration.

Monitoring Treatment

One skilled in the art is aware of means to monitor the therapeutic outcome and/or the systemic immune response upon administering a combination treatment of the present invention. In particular, the therapeutic outcome can be assessed by monitoring attenuation of tumor growth and/or tumor regression and or the level of tumor specific markers. The attenuation of tumor growth or tumor regression in response to treatment can be monitored using several end-points known to those skilled in the art including, for instance, number of tumors, tumor mass or size, or reduction/prevention of metastasis.

All literature and patent references cited above are hereby expressly incorporated by reference herein.

### EXAMPLE 1

Cytokine-Expressing Cellular Vaccines (GVAX) plus Anti-CTLA-4 Monoclonal Antibody

In vivo studies were carried out using the B16F10 model to determine if an anti-CTLA-4 mAb (9D9), in combination with a cytokine-expressing cellular vaccine (GVAX) can enhance anti-cancer efficacy. C57B16 mice were challenged with 1×10⁵ B16F10 tumor cells. One day later, mice were vaccinated with 3×10⁶ irradiated B16F10 or GM-CSF-secreting B16F10 (dB16gmtd) cells. One and three days after vaccination, mice were injected with 150 mg and 100 mg of anti-CTLA-4 (9D9 or 9H10) respectively. Mice received a second vaccination of 3×10⁶ irradiated cells two days after the last anti-CTLA-4 treatment. Mice were then monitored twice weekly for the development of subcutaneous tumors. Two anti-CTLA-4 mAbs, 9D9 (murine anti-CTLA4) and 9H10 (hamster anti-CTLA4) were tested in the B16 melanoma tumor model (FIG. 2A) and shown to have similar efficacy. The 9D9 anti-CTLA-4 mAb was tested in the B16 melanoma tumor model with and without vaccination with a cytokine-expressing cellular vaccine (GM-CSF-secreting B16F10 tumor cells; B16-GM) (FIG. 2B). The results show that the 9D9 anti-CTLA-4 mAb was ineffective as a monotherapy in protecting C57B1/6 mice from a B16F10 tumor challenge. In contrast, 80% of the mice treated with B16-GM plus 9D9 were protected, demonstrating that the 9D9 anti-CTLA4 mAb is biologically active and further supports the concept that anti-CTLA-4 and GVAX vaccines are an effective anti-cancer combination therapy (FIG. 2B).

### EXAMPLE 2

Cytokine-Expressing Cellular Vaccine (GVAX)+anti-41 BB mAb

In vivo studies were carried out using the B16F10 model to determine if an anti-41 BB monoclonal antibody in combination with a cytokine-expressing cellular vaccine (GVAX) can enhance anti-cancer efficacy.

On day 0 mice were challenged with 1×10⁵ live B16F10 (by sub-cu (SC) injection at a dorsal site in 0.5 ml volume). On day 3, mice were vaccinated with 1×10⁶ irradiated vaccine cells (as indicated in Table 2, below), followed by injection of 150 ug of anti-41BB mAb IP on day 6 (IP in 0.5 ml volume).

TABLE 2
B16F10 Tumor Model Treatment Groups For Combinations
Comprising anti-41BB Antibody

| Group # | # mice | Challenge | Vaccination | Treatment |
|---|---|---|---|---|
| 100 A-L | 12 | B16F10 | HBSS | None |
| 200 A-L | 12 | B16F10 | B16F10 | None |
| 300 A-L | 12 | B16F10 | GVAX | None |
| 400 A-L | 12 | B16F10 | GVAX | 41BB |

Mice were assessed daily for any obvious abnormality and if subcutaneous tumors reached 15-20 mm-diameter in size or started to ulcerate through the skin animals were be euthanized. On day 21, spleens and blood were collected and used for analysis of anti-tumor CTL, cytokine release (ELISA) and intracellular FACS.

The results indicate that the combined administration of GVAX and anti-4-1 BB antibody enhanced both the number of tumor free mice (FIG. 4A) and survival (FIG. 4B) relative to administration of GVAX alone, for up to 80 days post-challenge.

### EXAMPLE 3

Cytokine-Expressing Cellular Vaccine (GVAX)+CD40L

A variety of in vivo studies have been carried out to evaluate both the potential of cytokine-expressing cellular vaccine combination with anti-CD40 and CD40L as well as the parameters for optimizing the efficacy of such combinations. Factors to be considered in designing clinical studies include but are not limited to, relative timing of administration of the components of the combination, dose of each component, route and frequency of administration. In the case where the cytokine and additional cancer therapeutic agent are expressed by a cell, e.g., GM-CSF and CD40L, the vector for introduction into the cell must also be considered together with the level of expression by the cells and the type and manner of administration to the patient (i.e. autologous, allogeneic or bystander approach).

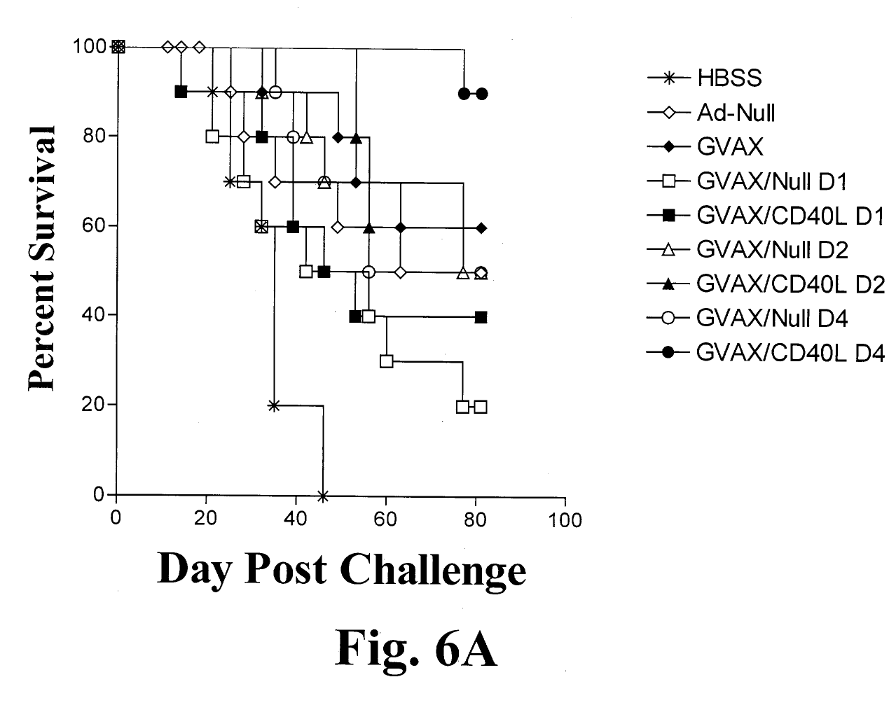

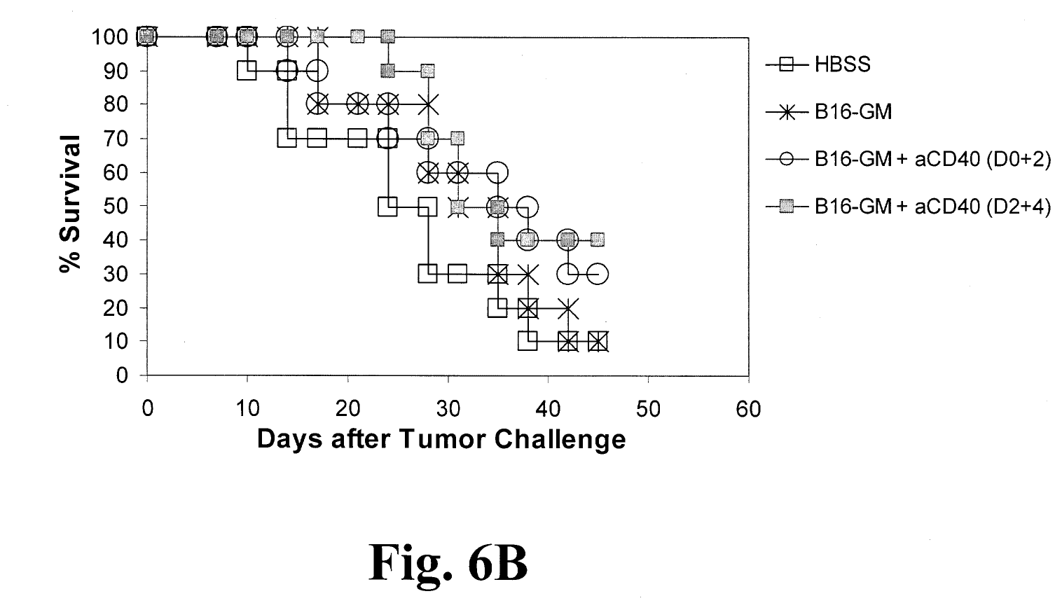

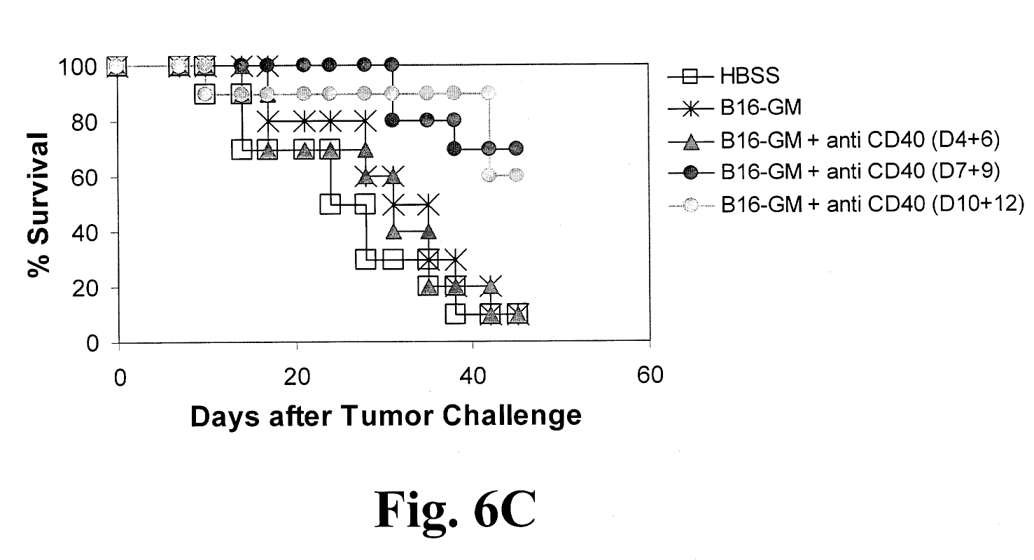

In vivo studies were carried out to determine if anti-CD40 or CD40L in combination with a cytokine-expressing cellular vaccine (GVAX) can enhance anti-cancer efficacy of a cytokine-expressing cellular vaccine alone (FIG. 5). In one exemplary experiment, Ad-NULL, Ad-GVAX (Ad-GM-CSF) and Ad-CD40L vaccines were prepared and tumor cells transduced using standard procedures, such as described in WO 00/72686, expressly incorporated by reference herein. In carrying out such experiments either separate populations of cells are transduced with each of Ad-GM-CSF and Ad-CD40L or the same population of cells is transduced with both vectors. If separate populations of cells are transduced with the individual vectors, the cell populations may be mixed and administered at the same time or administered at different times as described below. In this experiment, on day 0 mice in Groups 1-11 were challenged with 1×10⁵ live B16F10 mouse melanoma cells, (SC dorsal site in 0.5 ml volume). On day 3 after challenge, mice were vaccinated ventrally with 1×10⁶ B16-GM, followed by administration of further irradiated vaccine cells on day 3 (Group 5), day 4 (Groups 6 and 7), day 5 (Groups 8 and 9) and day 7 (Groups 10 and 11), as shown below in Table 3. Mice were monitored for tumor burden every 3-4 days for 3 months (n=10) and sacrificed on day 18, with spleens and blood collected for immunological assays. The results shown in FIG. 6A demonstrate that B16F10 cells transduced by an adenovirus vector encoding CD40 express CD40L, and when administered in combination with B16-GM, significantly increase survival of mice challenged with B16F10 melanoma, in particular when Ad-CD40L-transduced cells were administered 4 days after vaccination. When CD40L was administered the same day as B 16-GM, the disease was exacerbated.

TABLE 3
B16F10 Tumor Model Treatment Groups For
Combinations CD40 Ligand

| Group | # Mice | Vaccination |
|---|---|---|
| 1 | 12 | HBSS |
| 2 | 12 | Ad-Null (100 MOI) |
| 3 | 12 | Ad-GVAX (100 MOI) + Ad-Null (100 MOI) (1:1) |
| 4 | 12 | Ad-CD40L (100 MOI) + Ad-Null (100 MOI) (1:1) |
| 5 | 12 | Ad-GVAX (100 MOI) + Ad-CD40L (100 MOI) (1:1) |
| | | Day of GVAX |
| 6 | 12 | Ad-GVAX (100 MOI) + Ad-CD40L (100 MOI) (1:1) |
| | | Day 1 post GVAX |
| 7 | 12 | Ad-GVAX (100 MOI) + Ad-Null (100 MOI) (1:1) |
| | | Day 1 post GVAX |
| 8 | 12 | Ad-GVAX (100 MOI) + Ad-CD40L (100 MOI) (1:1) |
| | | Day 2 post GVAX |
| 9 | 12 | Ad-GVAX (100 MOI) + Ad-Null (100 MOI) (1:1) |
| | | Day 2 post GVAX |
| 10 | 12 | Ad-GVAX (100 MOI) + Ad-CD40L (100 MOI) (1:1) |
| | | Day 4 post GVAX |
| 11 | 12 | Ad-GVAX (100 MOI) + Ad-Null (100 MOI) (1:1) |
| | | Day 4 post GVAX |

MOI stands for multiplicity of infection which represents the number of infectious viral particles to tumor cells.

To further demonstrate the efficacy of B16-GM anti-CD40 combinations, animals were treated with an anti-CD40 monoclonal antibody administered at different times post vaccination. In this experiment, on day 0 mice were challenged with 1×10⁵ live B16F10 mouse melanoma cells, (SC dorsal site in 0.5 ml volume). On day 3 after challenge, mice were vaccinated ventrally with 1×10⁶ B16-GM. Anti-CD40 antibody was administered on days 0 and 2 or days 2 and 4 (FIG. 6B); days 4 and 6, days 7 and 9, or and days 10 and 12 (FIG. 6C). Mice were monitored for tumor burden every 3-4 days for 3 months (n=10) and sacrificed on day 18, with spleens and blood collected for immunological assays.

The results demonstrate that the combination of either membrane bound CD40L or monoclonal anti-CD40 antibody and B16-GM vaccine significantly enhances the efficacy of B16F10 vaccine expressing GM-CSF in the B16-GM melanoma model. The results from B16 melanoma model studies further show that the timing of CD40L and anti-CD40 administration is critical to efficacy (FIGS. 6A-6C).

### EXAMPLE 4

Cytokine-Expressing Cellular Vaccine (GVAX)+Interferon-Alpha

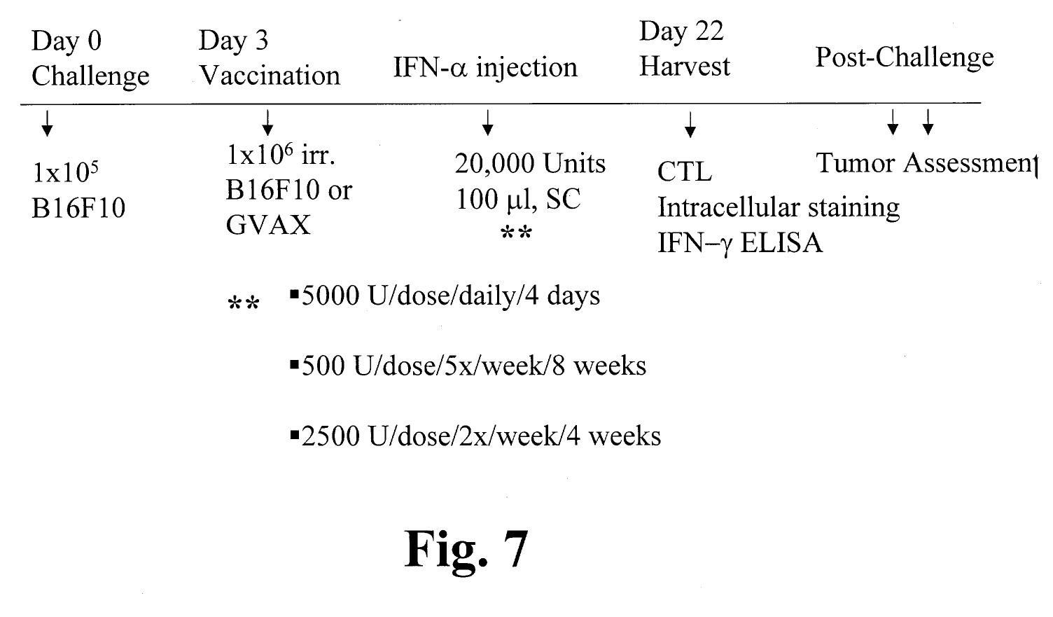

In vivo studies were carried out to determine if interferon-alpha in combination with a cytokine-expressing cellular vaccine (GVAX) can enhance anti-cancer efficacy. A schematic depiction of the experimental design is provided in FIG. 7.

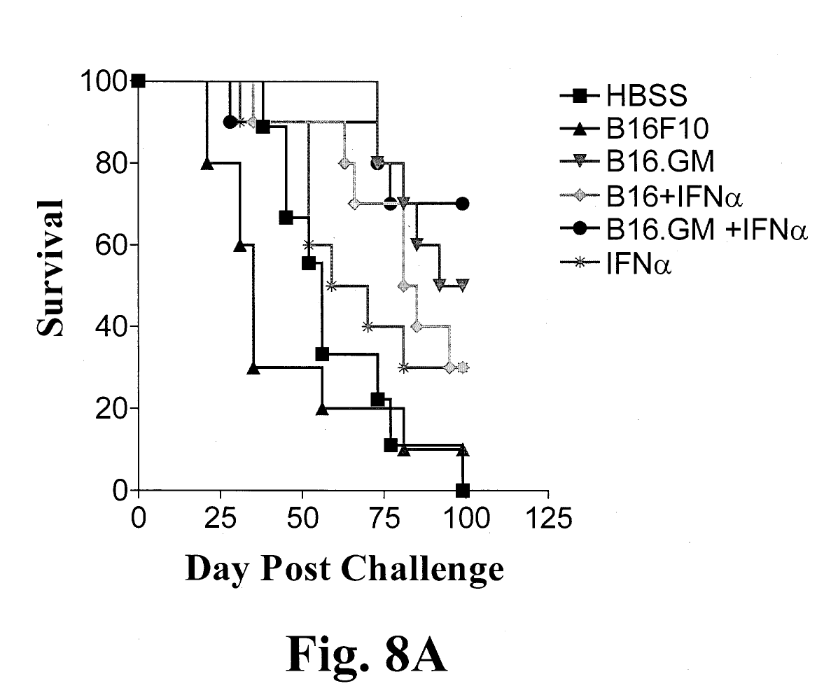

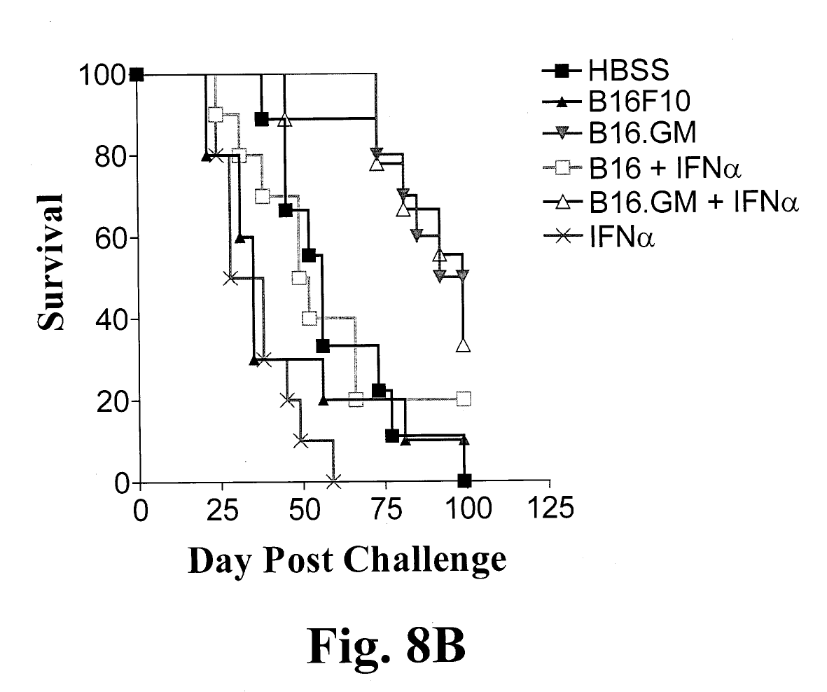

In an exemplary study, C57B16 mice were challenged with 1×10⁵ B16F10 tumor cells. Three days later, mice were vaccinated with 1×10⁶ irradiated B16F10 or GM-CSF-secreting B16F10 (B16.GM) cells. Four days after the vaccine, IFN-alpha administration was initiated. IFN-alpha (2500 units/day; twice/week) was injected subcutaneously at the site of tumor challenge (FIG. 8A) or the vaccination site (FIG. 8B). Mice were then monitored twice weekly for the development of subcutaneous tumors. The results show that recombinant murine IFN-alpha (rmIFN-alpha) given as a monotherapy offered minimal protection (approximately 30%) to mice challenged with B16F10 tumor cells when it was administered subcutaneously at the tumor challenge site, but no protection when administered at a distant site (FIGS. 8A and B). Approximately 50% of animals that received B16.GM alone remained tumor free after challenge (FIG. 8A). Administration of the combination of IFN alpha and B16.GM resulted in approximately 70% tumor free survival (FIG. 8A). These effects were observed following multiple IFN-alpha dosing regimens (20000 U total dose), including four daily injections of 5000 U or a metronomic regimen that consisted of 500 U/day, 5 days/week for 8 weeks.

In summary, the data demonstrate that IFN-alpha combined with a GM-CSF expressing cellular vaccine results in increased survival in the B16F10 model, and may represent a new approach to combination tumor immunotherapy.

### EXAMPLE 5

Cytokine-Expressing Cellular Vaccine (GVAX)+OX40/OX40L

The efficacy of a cytokine-expressing cellular vaccine (e.g., GVAX) and anti-OX40 or membrane bound/soluble OX40L may be evaluated using the B16F10 melanoma tumor model using the exemplary study design shown in Table 4, below.

TABLE 4
B16F10 Tumor Model Treatment Groups For
Combinations Comprising Anti-OX40 or
OX40L: Ig Fusion Protein

| GROUP | TREATMENT |
|---|---|
| 1 | HBSS only (negative control) |
| 2 | Irradiated BI6FI0/HBSS (control) |
| 3 | GVAX/HBSS (GVAX as monotherapy) |
| 4 | Anti-OX40 or OX40L: Ig fusion protein only as |
| 5 | Irradiated B16F10/OX40 stimulation (Combination of |
| 6 | Irradiated GVAX/OX40 (Combination GVAX and OX40 |

In optimizing the treatment protocol, a number of variables are important, such as outlined in Table 5, below.

TABLE 5
Combination therapy with dB16-retro GM-CSF
secreting cells and OX40.

| | | # per | # of | Total/ | # of | Total # |
|---|---|---|---|---|---|---|
| Combination | Variable | group | groups | Experiment | Expts. | of Mice |
| Anti-OX40 | Anti-OX40 | 12 | 6-10 | 72-120 | 2-3 | 144-360 |
| | dosed at the same | | | | | |
| | time as GVAX | | | | | |
| Anti-OX40 | Timing relative | 12 | 6-10 | 72-120 | 2-3 | 144-360 |
| | to GVAX | | | | | |
| Anti-OX40 | Single vs | 12 | 6-10 | 72-120 | 2-3 | 144-360 |
| | multiple GVAX plus | | | | | |
| | Anti-OX40 | | | | | |
| OX40L | Soluble vs | 12 | 6-10 | 72-120 | 2-3 | 144-360 |
| Expression | membrane | | | | | |
| | bound OX40L | | | | | |
| | on vaccine cells | | | | | |
| Anti-OX40 | Different tumor | 12 | 5 | 60 | 2-3 | 120-180 |
| | Models (CT26, | | | | | |
| | 4TI, etc) | | | | | |

Based on published literature and various experimental results, OX40-mediated protection ranges from 20-60% depending on the inherent immunogenicity of the tumor (Weinberg, A. et al., J. Immunol., 164: 2160-2169, 2000). The degree of anti-tumor protection afforded by the combination of GVAX and OX40 is dependent upon several factors such as timing relative to GVAX, the route of administration and the relative, dose of anti-OX40 or OX40L:Ig fusion protein and GVAX.

### EXAMPLE 7

Cytokine-Expressing Cellular Vaccine (GVAX)+Hsp70

The efficacy of a cytokine-expressing cellular vaccine (e.g., GVAX) and Hsp70 may be evaluated using the B16F1 melanoma tumor model. An exemplary study design follows:

TABLE 6
B16F10 Tumor Model Treatment Groups For
Combinations Comprising hsp70

| GROUP | TREATMENT |
|---|---|
| 1 | HBSS only (negative control) |
| 2 | Irradiated BI6FI0/HBSS (control) |
| 3 | GVAX/HBSS (GVAX as monotherapy) |
| 4 | Irradiated B16F10/hsp70 (hsp 70 as monotherapy) |
| 5 | Irradiated GVAX/hsp70 (Combination GVAX/hsp70) |

In optimizing the treatment protocol, a number of variables are important, such as outlined in Table 7, below.

TABLE 7
Combination therapy with dB16-retro GM-CSF
secreting: cells and Hsp70

| | | # per | # of | Total/ | # of | Total # of |
|---|---|---|---|---|---|---|
| Combination | Variable | group | groups | Expt. | Expts. | Mice |
| hsp70 | Dose of hsp70 at the | 12 | 6-10 | 72-120 | 2-3 | 144-360 |
| | same time as GVAX | | | | | |
| hsp70 | Timing of hsp70 | 12 | 6-10 | 72-120 | 2-3 | 144-360 |
| | relative to GVAX | | | | | |
| hsp70 | Single vs multiple | 12 | 6-10 | 72-120 | 2-3 | 144-360 |
| | doses of GVAX plus hsp70 | | | | | |

The degree of anti-tumor protection afforded by the combination of GVAX and hsp70 will be dependent upon several factors such as timing of HSP administration relative to that of GVAX, the route of administration and the relative dose of GVAX versus the dose of hsp70.

1. An improved method of cancer immunotherapy, the improvement comprising:
administering the combination of a cytokine-expressing cellular vaccine and at least one additional cancer therapeutic agent selected from the group consisting of an anti-CTLA4 antibody, an anti-4-1BB antibody, an anti-CD40 antibody or CD40 ligand, interferon-alpha, IL-2, an anti-OX40 antibody or OX-40 ligand, CpC oligonucleotide, and paclitaxel and docetaxel to a subject with cancer, wherein administration of the combination results in enhanced therapeutic efficacy relative to administration of the cytokine-expressing cellular vaccine or the at least one additional cancer therapeutic agent alone.

2. The method of claim 1, wherein the cytokine-expressing cellular vaccine expresses GM-CSF.

3. The method of claim 2, wherein the combination comprises cells that are autologous to the subject.

4. The method of claim 2, wherein the combination comprises cells that are allogeneic to the subject.

5. The method of claim 2, wherein the comprises bystander cells.

6. The method of claim 2, wherein the cytokine-expressing cellular vaccine is rendered proliferation-incompetent by irradiation.

7. The method of claim 2, wherein the mammal is a human.

8. The method of claim 2, wherein the cancer is a prostate cancer.

9. The method of claim 2, wherein the cancer is a non-small cell lung carcinoma.

10. The method of claim 4, wherein the allogeneic cells are a tumor cell line selected from the group consisting of a prostate tumor line, a non-small cell lung carcinoma line and a pancreatic cancer line.

11. The method of claim 2, wherein the at least one additional cancer therapeutic agent is an antibody selected from the group consisting of an anti-CTLA4 antibody, an anti-4-1BB antibody, an anti-CD40 antibody and an anti-OX40 antibody.

12. The method of claim 11, wherein the antibody is an anti-CTLA4 antibody.

13. The method of claim 11, wherein the antibody is an anti-4-1BB antibody.

14. The method of claim 11, wherein the antibody is an anti-CD40 antibody.

15. The method of claim 2, wherein the at least one additional cancer therapeutic agent is selected from the group consisting of a CD40 ligand, interferon-alpha, IL-2, OX-40 ligand and a CpG oligonucleotide.

16. The method of claim 15, wherein the at least one additional cancer therapeutic agent is expressed by a cell and the cell is autologous.

17. The method of claim 15, wherein the at least one additional cancer therapeutic agent is expressed by a cell and the cell is allogeneic.

18. The method of claim 15, wherein the at least one additional cancer therapeutic agent is expressed by a cell and the cell is a bystander cell.

19. The method of claim 15, wherein the at least one additional cancer therapeutic agent is CD40 ligand.

20. The method of claim 15, wherein the at least one additional cancer therapeutic agent is interferon-alpha.

21. The method of claim 15, wherein the at least one additional cancer therapeutic agent is OX-40 ligand.

22. The method of claim 15, wherein the at least one additional cancer therapeutic agent is a CpG oligonucleotide.

23. The method of claim 2, wherein the cytokine-expressing cellular vaccine is administered subcutaneously.

24. The method of claim 2, wherein the cytokine-expressing cellular vaccine is administered intratumorally.

25. The method of claim 14, wherein the anti-CD40 antibody is administered after the GM-CSF-expressing cellular vaccine.

26. The method of claim 19, wherein CD40 ligand is administered after the GM-CSF-expressing cellular vaccine.

27. The method of claim 2, wherein the at least one additional cancer therapeutic agent is paclitaxel or docetaxel.

28. The method of claim 2, wherein the at least one additional cancer therapeutic agent is paclitaxel.

29. The method of claim 2, wherein the at least one additional cancer therapeutic agent is docetaxel.

30. The method of claim 28, wherein said paclitaxel is administered after said GM-CSF-expressing cellular vaccine.

31. The method of claim 29, wherein said docetaxel is administered after said GM-CSF-expressing cellular vaccine.

32. The method of claim 28, wherein said paclitaxel is administered before said GM-CSF-expressing cellular vaccine.

33. The method of claim 29, wherein said docetaxel is administered before said GM-CSF-expressing cellular vaccine.
